(19) United States
(12) Patent Application Publication
(10) Pub. No.: US 2014/0213925 A1
(43) Pub. Date: Jul. 31, 2014

(54) SYSTEMS, METHODS AND KITS FOR MEASURING RESPIRATORY RATE AND DYNAMICALLY PREDICTING RESPIRATORY EPISODES

(71) Applicant: ISONEA LIMITED, Armadale, Victoria (AU)

(72) Inventors: Johnny Yat Ming Chan, Newport Beach, CA (US); Stephen Anthony Tunnell, Oceanside, CA (US); Michael Joseph Thomas, Severna Park, MD (US)

(21) Appl. No.: 14/342,383

(22) PCT Filed: Sep. 20, 2012
(86) PCT No.: PCT/US12/56293

§ 371 Date: Mar. 2, 2014

Related U.S. Application Data
(60) Provisional application No. 61/536,841, filed on Sep. 20, 2011.

(51) Int. Cl. A61B 5/00; A61B 7/00; A61B 5/091; A61B 5/087; A61B 5/097

(52) U.S. Cl. CPC A61B 5/7275; A61B 5/0871; A61B 5/097; A61B 5/091; A61B 5/742; A61B 5/7282; A61B 5/7405; A61B 7/003; A61B 5/0004; A61B 5/0022
USPC 600/538; 600/586; 600/529; 600/595

(57) ABSTRACT

This disclosure is directed to devices, systems, kits and methods for measuring peak expiratory or inspiratory flow-rate and dynamically predicting respiratory episodes. Additionally, systems for analyzing and processing the measurement in a communication networked environment are also provided. An aspect of the disclosure is directed to a respiratory device, In some configurations the respiratory device comprises a housing adaptable and configurable to communicate with an electronic device, a mouth piece having a proximal end and a distal end configurable to engage a mouth of a patient and transmit an air flow, one or more diaphragm sensors configured to detect a breath vibration from the air flow in the mouth piece, and a processor adaptable and configurable to analyze the breath vibration detected by the one or more diaphragm sensors.

### CROSS-REFERENCE

This application claims the benefit of U.S. Provisional Application No. 61/536,841, filed Sep. 20, 2011, entitled Systems, Methods and Kits for Measuring Respiratory Rate, by Chan, Tunnell and Thomas, which application is incorporated herein by reference.

### BACKGROUND OF THE INVENTION

Asthma is an episodic chronic disease that disrupts normal respiratory function in mammals. One aspect of asthma therapy involves preventing episodes of extreme worsening of respiratory function such as those associated with asthma attacks. During an asthma attack, or asthma exacerbation, the patient's airways become swollen and inflamed. Additionally, muscles associated with the patient's airways contract which causes the bronchial tubes to narrow. Patients will often wheeze, cough, and have trouble breathing. The severity of the attack can be minor or result in a life threatening emergency requiring a trip to the hospital.

The US National Institutes of Health (NIH) has recommended that asthma suffers take an Asthma Control Test (ACT) to assess the level of control of asthma. ACT is a tool that patients and healthcare providers use to assess asthma conditions and control. Patients answer a series of questions which look back over a period of time to assess whether shortness of breath was experienced, number of times a patient awoke during their sleep cycle from asthma relates symptoms (shortness of breath, chest tightness, or pain), the number of times a rescue inhaler was used (such as albuterol), as well as a subjective personal rating of the patient's impression of control over the same period of time.

The peak flow meter was invented by a British bioengineer, Basil Martin Wright (1912-2001) to provide a useful measurement to manage asthma symptoms. Peak Flow Meters work by mechanically measuring how fast air comes out of the lungs when a patient exhales forcefully after inhaling fully. This measurement is referred to as a “Peak Expiratory Flow” (PEF). Keeping track of PEF is one way a patient could monitor and understand if his/her asthma symptoms are controlled or worsening.

A classic flow metering system called Variable Area Orifice Metering (VAOM) is one type of system that is used for flow measurement. See, e.g., Wright B M, McKerrow C B. Maximum forced expiratory flow-rate as a measure of ventilator capacity. BMJ 1959; ii: 1043. VAOM is one of the flow measurement methods defined in Mechanical Engineering field. Wright applied this methodology on peak expiratory flow measurement application and became the first peak flow meter.

Wright later applied rotameter, an advanced form of VAOM in his Peak Flow Meter. A rotameter contains a tapered metering elongated tube with a float positioned therein. When the air or fluid flows through the metering tube, a force will be generated against or opposing gravity and push the float up. A mechanical rider will mark the highest equilibrium the float reached, also known as the maximum flow-rate.

Wright adapted this method and applied on the peak-flow metering. Instead of relying solely on the gravity, Wright increased the tension by adding a mechanical spring. As described in his article, exhale air flows through the tapered metering tube and push a piston, which rides freely on the central rod (the float), against the attached mechanical spring. A rider being pushed by the piston marks the maximum equilibrium.

Currently, there are many peak-flow meters available in market, all of which rely on the mechanical principles established by Wright. See, for example, U.S. Design Pat. Des. 332,229 for Peak Flow Meter issued Jan. 5, 1993, to Brown, and Des. 586,248 for Peak Flow Meter issued Feb. 10, 2009, to Baker; and U.S. Pat. No. 6,889,564 B1 for Peak Flow Meter issued May 10, 2005, to Marcotte et al., and U.S. Pat. No. 3,862,628 for Peak Flow Meters issued Jan. 28, 1975, to Williams. Other references relating to asthma include, for example, U.S. Pat. No. 8,231,541 B2 for Asthma Status Scoring Method and System with Confidence Ratings issued Jul. 31, 2012 to Colquitt et al.

What is needed is: a peak expiratory flow-rate device that can take measurements electronically; a flow-rate device that is configurable to operate as part of a communication network; a respiratory functionality system assessment and predictor; and a method or system that can determine the likelihood of a respiratory incident, such as an asthma attack, prior to its onset and which is configurable to operate as part of a communication network.

### SUMMARY OF THE INVENTION

Devices, systems and methods for obtaining higher accuracy peak flow measurements are disclosed. Users and health care practitioners can keep track of measurements from a peak flow meter, such as those disclosed, by using the networked systems and methods for tracking.

An aspect of the disclosure is directed to a respiratory device. In some configurations the respiratory device comprises: a housing adaptable and configurable to communicate with an electronic device (e.g. a form factor that is configurable to fit over or attaches to a portion of the form factor of the electronic device); a mouth piece having a proximal end and a distal end configurable to engage a mouth of a patient and transmit an air flow; one or more diaphragm sensors configured to detect a breath vibration from the air flow in the mouth piece; and a processor adaptable and configurable to analyze the breath vibration detected by the one or more diaphragm sensors. The respiratory device is also adaptable and configurable to measure one or more patient flow-rate data including, for example, of peak expiratory flow-rate, peak inspiratory flow-rate, mean flow-rates, volumes, flow over time, Forced Vital Capacity, percentage of flow at certain time intervals, and slow and forced volumes at certain time intervals, the device. The respiratory device is also configurable to be in communication with a display adapted and configured to display a result of an analysis of the breath vibration. In at least some configurations a windscreen can be provided which is positionable relative to the mouth piece to reduce a secondary air flow from entering the mouth piece. The device is also configurable to be in communication with an environmental data source to obtain environmental data, including, but not limited to, one or more of each of an environmental sensor associated with one or more of the respiratory device and the electronic device. In some configurations the processor is further adaptable and configurable to correlate breath vibration data and environmental data. Additionally, the processor is adaptable and configurable to generate an asthma output. In other configurations the respiratory device is adaptable and configurable to be in communication with an audible indicator. Additionally, users can provide active or passive input. Active input occurs when the user enters information concerning, for example, current conditions, medications, etc. Passive input occurs when the system logs that the user took an action, such as using the device.

Another aspect of the disclosure is directed to a respiratory rate measuring system. The system is adaptable and configurable to comprise: measurement probe, the probe comprising; one or more diaphragm sensors adaptable and configurable to detect a breath vibration; a microphone; a mouth piece positioned proximally to the microphone; and a port, a computing system adaptable to communicate with the port and having a computer executable instruction that, when executed by a processor, performs a vibration analysis of the diaphragm vibration and display the result. In some configurations, the system is adaptable and configurable to measure one or more of peak expiratory flow-rate, peak inspiratory flow-rate, mean flow-rates, volumes, flow over time, Forced Vital Capacity, percentage of flow at certain time intervals, and slow and forced volumes at certain time intervals, the device. Additionally, the respiratory device is adaptable and configurable to be in communication with a display adapted and configured to display a result of an analysis of the breath vibration. In some configurations, a windscreen is provided which is positionable relative to the mouth piece to reduce a secondary air flow from entering the mouth piece. In still other configurations, the system is in communication with an environmental data source, such as an environmental sensor associated with one or more of the respiratory device and the electronic device. Additionally, the processor is further adaptable and configurable to correlate breath vibration data and environmental data. The processor is also adaptable and configuration to generate an asthma output. In still other configurations, the respiratory device is in communication with an audible indicator. The computing systems are selectable from the group comprising a computer, a mobile phone, a smart phone, a handheld device.

Yet another aspect of the disclosure is directed to a system comprising: an electronic device having a microphone adaptable and configurable to be in communication with a communication network; a housing attachable to the electronic device, the housing further comprising: a mouth piece aligned with a microphone of the electronic device; and an optional windscreen resided in a mouth piece tube; a computer executable instruction that, when executed by a processor performs operations including vibration analysis of the microphone diaphragm of the probe and further adaptable to instruct that a result of the vibration analysis be displayed on a display. In some configurations, the system is adaptable and configurable to measure one or more of peak expiratory flow-rate, peak inspiratory flow-rate, mean flow-rates, volumes, flow over time, Forced Vital Capacity, percentage of flow at certain time intervals, and slow and forced volumes at certain time intervals, the device. Additionally, the respiratory device is adaptable and configurable to be in communication with a display adapted and configured to display a result of an analysis of the breath vibration. In some configurations, a windscreen is provided which is positionable relative to the mouth piece to reduce a secondary air flow from entering the mouth piece. In still other configurations, the system is in communication with an environmental data source, such as an environmental sensor associated with one or more of the respiratory device and the electronic device. Additionally, the processor is further adaptable and configurable to correlate breath vibration data and environmental data. The processor is also adaptable and configuration to generate an asthma output. In still other configurations, the respiratory device is in communication with an audible indicator. The computing systems are selectable from the group comprising a computer, a mobile phone, a smart phone, a handheld device.

In still another aspect of the disclosure, non-transitory computer readable medium storing instructions that, when executed by a computing device, causes the computing device to perform a method, the method comprising: receiving one or more of each of a peak expiratory flow-rate, a peak inspiratory flow-rate, and a spirometry measurement from a respiratory device having a housing adaptable and configurable to communicate with an electronic device to receive a user respiratory input; at least one or more of analyzing, monitoring, evaluating, and responding to the received one or more of the peak expiratory flow-rate, the peak inspiratory flow-rate, and the spirometry measurement, is provided. In at least some configurations, the method performed by the medium can further comprise one or more of each of the steps of: communicating with a remote server; one or more of recording data and transmitting data; receiving data from a secondary measurement device (such as data from a heart rate monitor, a heart sound sensor, and saturation of oxygen in arterial blood flow (SpO2 or pulse oximetry) data), determining a GPS location for the measurement, acquiring environmental data (such as one or more of each of pollen count, air pollution data, airborne particulate matter data, airborne irritants data ambient temperature, temperature changes, and humidity data), acquiring behavioral data (such as one or more of each of data for compliance with medication protocol, compliance with testing protocol, compliance with monitoring protocol, compliance with system generated recommendations, compliance with health care provider recommendations).

Still another aspect of the disclosure is directed to a computing device comprising: a processor configured to: receive one or more of each of a peak expiratory flow-rate, a peak inspiratory flow-rate, and a spirometry measurement from respiratory device having a housing adaptable and configurable to communicate with an electronic device to receive a user respiratory input; at least one or more of analyze, monitor, evaluate, and respond to the received one or more of the peak expiratory flow-rate, the peak inspiratory flow-rate, and the spirometry measurement. In at least some configurations the processor is adaptable and configurable to perform one or more of the following: communicate with a remote server; record data; transmit data; receive data from a secondary measurement device (such as data from a heart rate monitor, a heart sound sensor, and saturation of oxygen in arterial blood flow (SpO2 or pulse oximetry) data); determine a GPS location for the measurement, acquire environmental data (such as one or more of each of pollen count, air pollution data, airborne particulate matter data, airborne irritants data ambient temperature, temperature changes, and humidity data); acquire behavioral data (which could be one or more of each of data for compliance with medication protocol, compliance with testing protocol, compliance with monitoring protocol, compliance with system generated recommendations, compliance with health care provider recommendations).

In yet other aspects of the disclosure, the disclosure provides methods comprising: receiving one or more of each of a peak expiratory flow-rate, a peak inspiratory flow-rate, and a spirometry measurement from a respiratory device having a housing adaptable and configurable to communicate with an electronic device to receive a user respiratory input; receiving an environmental data input; at least one or more of analyzing, monitoring, evaluating, and responding to the received one or more of the peak expiratory flow-rate, the peak inspiratory flow-rate, and the spirometry measurement. The methods are also adaptable and configurable to include one or more the following steps: communicating with a remote server; recording data; transmitting data; receiving data from a secondary measurement device (such as data from a heart rate monitor, a heart sound sensor, and saturation of oxygen in arterial blood flow (SpO2 or pulse oximetry) data); determining a GPS location for the measurement; acquiring environmental data (such as one or more of each of pollen count, air pollution data, airborne particulate matter data, airborne irritants data ambient temperature, temperature changes, and humidity data); acquiring behavioral data (including, but not limited to one or more of each of data for compliance with medication protocol, compliance with testing protocol, compliance with monitoring protocol, compliance with system generated recommendations, compliance with health care provider recommendations).

Still another aspect of the disclosure is directed to a method comprising: transmitting, via a user computing device one or more of each of a peak expiratory flow-rate, a peak inspiratory flow-rate, and a spirometry measurement from a respiratory device having a housing adaptable and configurable to communicate with an electronic device to receive a user respiratory input to a web-server over a network; obtaining an environmental data input; at least one or more of analyzing, monitoring, evaluating, and responding to the received one or more of the peak expiratory flow-rate, the peak inspiratory flow-rate, and the spirometry measurement. The methods are also adaptable and configurable to include one or more the following steps: communicating with a remote server; recording data; transmitting data; receiving data from a secondary measurement device (such as data from a heart rate monitor, a heart sound sensor, and saturation of oxygen in arterial blood flow (SpO2 or pulse oximetry) data); determining a GPS location for the measurement; acquiring environmental data (such as one or more of each of pollen count, air pollution data, airborne particulate matter data, airborne irritants data ambient temperature, temperature changes, and humidity data); acquiring behavioral data (including, but not limited to one or more of each of data for compliance with medication protocol, compliance with testing protocol, compliance with monitoring protocol, compliance with system generated recommendations, compliance with health care provider recommendations).

Yet another aspect of the disclosure is directed to a system comprising: an electronic device configurable to be in communication with a communication network; a computer executable instruction that, when executed by a processor determines a likelihood of a respiratory event based on one or more of each of historical patient data, patient data input, current environmental data, current data for other patients in a similar geographic location and historical data for other patients in a similar geographic location. In some configurations, the processor is further adaptable and configurable to, one or more of, communicate with a remote server; record data and transmit data; receive data from a secondary measurement device (such as data from a heart rate monitor, a heart sound sensor, and saturation of oxygen in arterial blood flow (SpO2 or pulse oximetry) data); determine a GPS location for the measurement; acquire environmental data (such as one or more of each of pollen count, air pollution data, airborne particulate matter data, airborne irritants data ambient temperature, temperature changes, and humidity data); acquire behavioral data (including, but not limited to one or more of each of data for compliance with medication protocol, compliance with testing protocol, compliance with monitoring protocol, compliance with system generated recommendations, compliance with health care provider recommendations).

In still other aspects of the disclosure, a non-transitory computer readable medium storing instructions that, when executed by a computing device, causes the computing device to perform a method, the method comprising: receiving one or more of a GPS location and a condition indication for each of one or more patients comprising a patient group; at least one or more of analyzing, monitoring, evaluating, and providing a prediction for a second patient based on the GPS location of the second patient and at least one or more of the GPS location and condition indication for the one or more patients comprising the patient group. In some configurations, the processor is further adaptable and configurable to, one or more of, communicate with a remote server; record data and transmit data; receive data from a secondary measurement device (such as data from a heart rate monitor, a heart sound sensor, and saturation of oxygen in arterial blood flow (SpO2 or pulse oximetry) data); determine a GPS location for the measurement; acquire environmental data (such as one or more of each of pollen count, air pollution data, airborne particulate matter data, airborne irritants data ambient temperature, temperature changes, and humidity data); acquire behavioral data (including, but not limited to one or more of each of data for compliance with medication protocol, compliance with testing protocol, compliance with monitoring protocol, compliance with system generated recommendations, compliance with health care provider recommendations).

Still other aspects of the disclosure are directed to a computing device comprising: a processor configured to: receive one or more of GPS location and condition indication for each of one or more patients comprising a patient group; at least one or more of analyze, monitor, evaluate, and provide a prediction for a second patient based on the GPS location and at least one or more of the GPS location and condition indication for the one or more patients comprising the patient group. In some configurations, the processor is further adaptable and configurable to, one or more of, communicate with a remote server; record data and transmit data; receive data from a secondary measurement device (such as data from a heart rate monitor, a heart sound sensor, and saturation of oxygen in arterial blood flow (SpO2 or pulse oximetry) data); determine a GPS location for the measurement; acquire environmental data (such as one or more of each of pollen count, air pollution data, airborne particulate matter data, airborne irritants data ambient temperature, temperature changes, and humidity data); acquire behavioral data (including, but not limited to one or more of each of data for compliance with medication protocol, compliance with testing protocol, compliance with monitoring protocol, compliance with system generated recommendations, compliance with health care provider recommendations).

Another aspect of the disclosure is directed to a method comprising: receiving one or more of a GPS location and a condition indication for each of one or more patients comprising a patient group; at least one or more of analyzing, monitoring, evaluating, and providing a prediction for a second patient based on the GPS location of the second patient and at least one or more of the GPS location and the condition indication for the one or more patients comprising the patient group. The methods are also adaptable and configurable to include one or more the following steps: communicating with a remote server; recording data; transmitting data; receiving data from a secondary measurement device (such as data from a heart rate monitor, a heart sound sensor, and saturation of oxygen in arterial blood flow (SpO2 or pulse oximetry) data); determining a GPS location for the measurement; acquiring environmental data (such as one or more of each of pollen count, air pollution data, airborne particulate matter data, airborne irritants data ambient temperature, temperature changes, and humidity data); acquiring behavioral data (including, but not limited to one or more of each of data for compliance with medication protocol, compliance with testing protocol, compliance with monitoring protocol, compliance with system generated recommendations, compliance with health care provider recommendations).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A is a block diagram showing a representative example of a logic device through which peak flow-rate measurement and management can be achieved;

FIG. 1B is a block diagram of an exemplary computing environment through which peak flow-rate measurement and management can be achieved;

FIG. 1C is an illustrative architectural diagram showing some structure that can be employed by devices through which peak flow-rate measurement and management is achieved;

FIG. 2 is a block diagram showing the cooperation of exemplary components of a system suitable for use in a system where peak flow-rate measurement and management is achieved;

FIG. 3 illustrates an operating principle of a condenser microphone, suitable for use in devices of the disclosure;

FIGS. 4A-G illustrates a flow-rate detection device adapted and configured to measure inspiratory and/or expiratory flow from a patient adaptable to be in communication with a secondary electronic device;

FIGS. 5A-G illustrates a flow-rate detection device adapted and configured to measure inspiratory and/or expiratory flow from a patient adaptable to be in communication with a secondary electronic device;

FIG. 6-6D illustrates an alternative embodiment of a flow-rate detection device adapted and configured to measure inspiratory and/or expiratory flow from a patient;

FIGS. 7A-G illustrates a flow-rate detection device adapted and configured to measure inspiratory and/or expiratory flow from a patient adaptable to be in communication with a secondary electronic device;

FIG. 8 illustrates a flow-rate detection device adapted and configured to measure inspiratory and/or expiratory flow from a patient adaptable to be in communication with a secondary electronic device; and

FIG. 9 illustrates an interrelationship between data components in the system.

### DETAILED DESCRIPTION OF THE INVENTION

### I. Computing Systems

The systems and methods described herein rely on a variety of computer systems, networks and/or digital devices for operation. In order to fully appreciate how the system operates an understanding of suitable computing systems is useful. Aspects of the systems and methods disclosed herein can be enabled as a result of application via a suitable computing system.

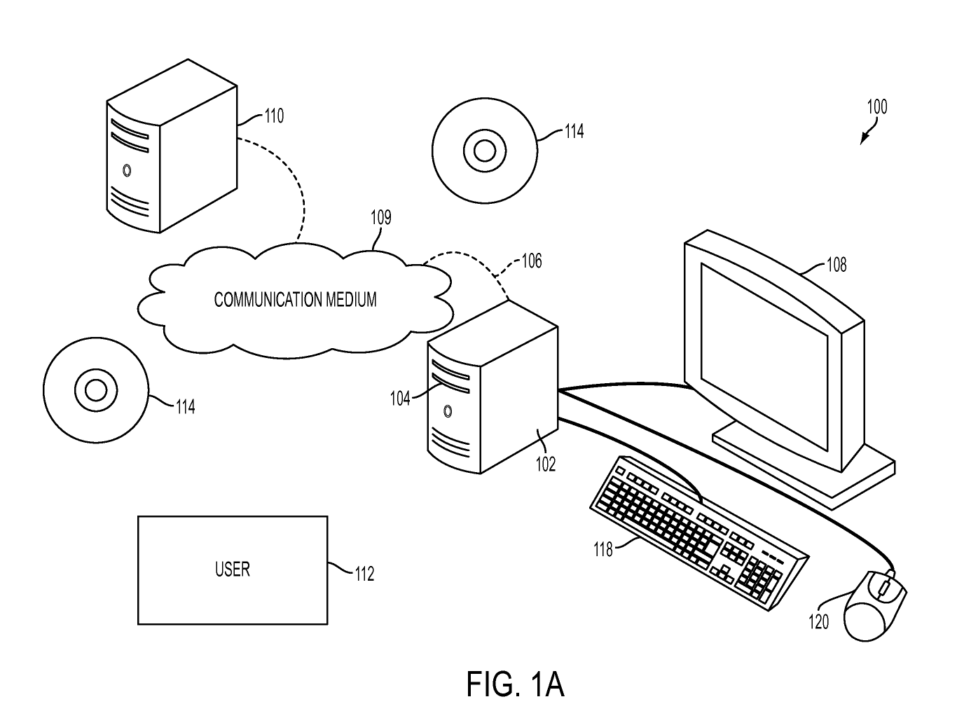

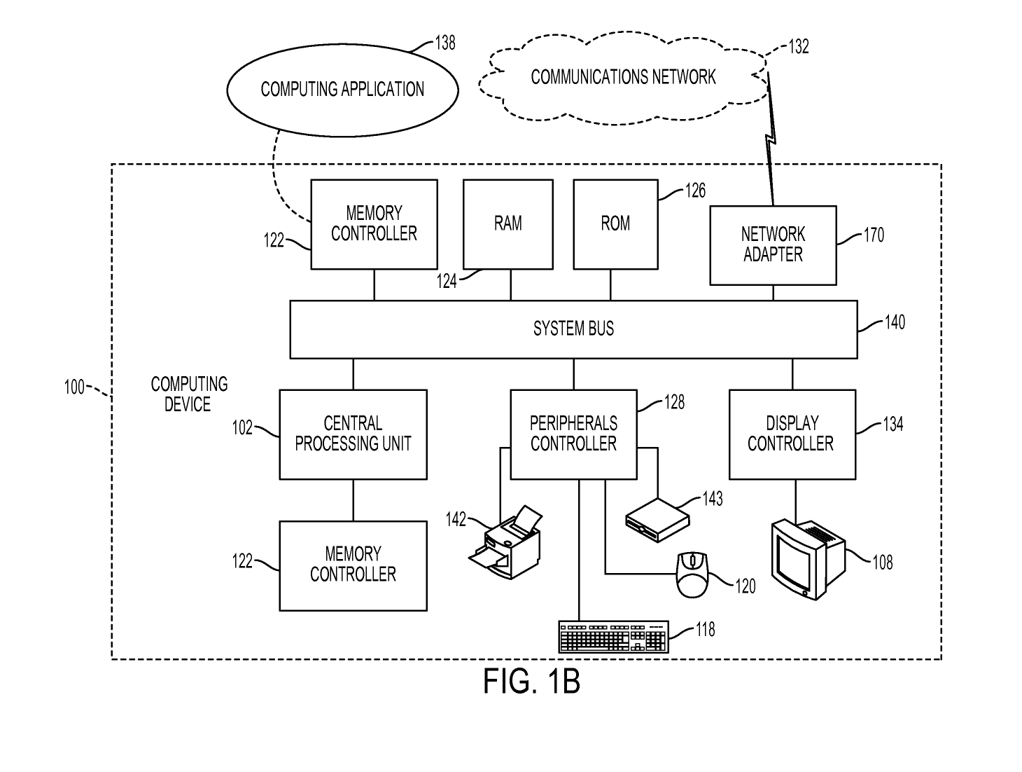

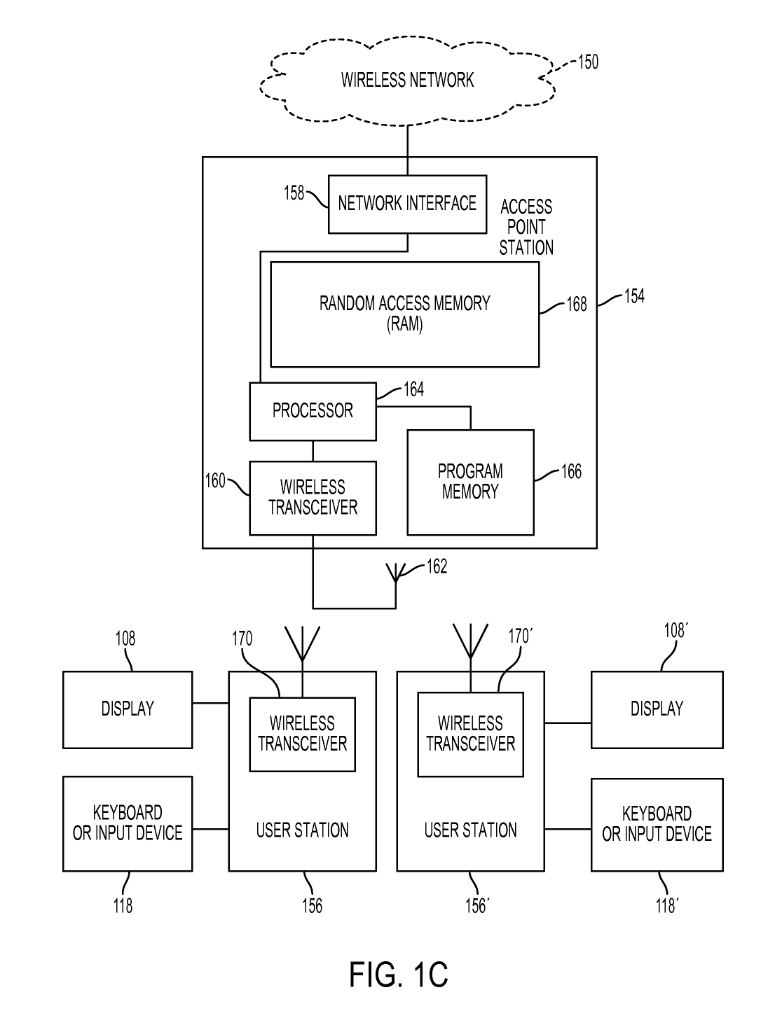

FIG. 1A is a block diagram showing a representative example logic device through which a browser can be accessed to implement the present invention. A computer system (or digital device) 100, which may be understood as a logic apparatus adapted and configured to read instructions from media 114 and/or network port 106, is connectable to a server 110, and has a fixed media 116. The computer system 100 can also be connected to the Internet or an intranet. The system includes central processing unit (CPU) 102, disk drives 104, optional input devices, illustrated as keyboard 118 and/or mouse 120 and optional monitor 108. Data communication can be achieved through, for example, communication medium 109 to a server 110 at a local or a remote location. The communication medium 109 can include any suitable means of transmitting and/or receiving data. For example, the communication medium can be a network connection, a wireless connection or an internet connection. It is envisioned that data relating to the present disclosure can be transmitted over such networks or connections. The computer system can be adapted to communicate with a participant and/or a device used by a participant. The computer system is adaptable to communicate with other computers over the Internet, or with computers via a server.

FIG. 1B depicts another exemplary computing system 100. The computing system 100 is capable of executing a variety of computing applications 138, including computing applications, a computing applet, a computing program, or other instructions for operating on computing system 100 to perform at least one function, operation, and/or procedure. Computing system 100 is controllable by computer readable storage media for tangibly storing computer readable instructions, which may be in the form of software. The computer readable storage media adapted to tangibly store computer readable instructions can contain instructions for computing system 100 for storing and accessing the computer readable storage media to read the instructions stored thereon themselves. Such software may be executed within CPU 102 to cause the computing system 100 to perform desired functions. In many known computer servers, workstations and personal computers CPU 102 is implemented by micro-electronic chips CPUs called microprocessors. Optionally, a co-processor, distinct from the main CPU 102, can be provided that performs additional functions or assists the CPU 102. The CPU 102 may be connected to co-processor through an interconnect. One common type of coprocessor is the floating-point coprocessor, also called a numeric or math coprocessor, which is designed to perform numeric calculations faster and better than the general-purpose CPU 102.

As will be appreciated by those skilled in the art, a computer readable medium stores computer data, which data can include computer program code that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable storage media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other physical or material medium which can be used to tangibly store the desired information or data or instructions and which can be accessed by a computer or processor.

Some embodiments may be implemented in one or a combination of hardware, firmware and software. Embodiments may also be implemented as instructions stored on a non-transitory computer-readable storage medium, which may be read and executed by at least one processor to perform the operations described herein. A non-transitory computer-readable storage medium may include any mechanism for storing information in a form readable by a machine (e.g., a computer). For example, a non-transitory computer-readable storage medium may include read-only memory (ROM), random-access memory (RAM), magnetic disk storage media, optical storage media, flash-memory devices, and other non-transitory media.

In operation, the CPU 102 fetches, decodes, and executes instructions, and transfers information to and from other resources via the computer's main data-transfer path, system bus 140. Such a system bus connects the components in the computing system 100 and defines the medium for data exchange. Memory devices coupled to the system bus 140 include random access memory (RAM) 124 and read only memory (ROM) 126. Such memories include circuitry that allows information to be stored and retrieved. The ROMs 126 generally contain stored data that cannot be modified. Data stored in the RAM 124 can be read or changed by CPU 102 or other hardware devices. Access to the RAM 124 and/or ROM 126 may be controlled by memory controller 122. The memory controller 122 may provide an address translation function that translates virtual addresses into physical addresses as instructions are executed.

In addition, the computing system 100 can contain peripherals controller 128 responsible for communicating instructions from the CPU 102 to peripherals, such as, printer 142, keyboard 118, mouse 120, and data storage drive 143. Display 108, which is controlled by a display controller 163, is used to display visual output generated by the computing system 100. Such visual output may include text, graphics, animated graphics, and video. The display controller 134 includes electronic components required to generate a video signal that is sent to display 108. Further, the computing system 100 can contain network adaptor 136 which may be used to connect the computing system 100 to an external communications network 132.

### II. Networks and Internet Protocol

As is well understood by those skilled in the art, the Internet is a worldwide network of computer networks. Today, the Internet is a public and self-sustaining network that is available to many millions of users. The Internet uses a set of communication protocols called TCP/IP (i.e., Transmission Control Protocol/Internet Protocol) to connect hosts. The Internet has a communications infrastructure known as the Internet backbone. Access to the Internet backbone is largely controlled by Internet Service Providers (ISPs) that resell access to corporations and individuals.

The Internet Protocol (IP) enables data to be sent from one device (e.g., a phone, a Personal Digital Assistant (PDA), a computer, etc.) to another device on a network. There are a variety of versions of IP today, including, e.g., IPv4, IPv6, etc. Other IPs are no doubt available and will continue to become available in the future, any of which can be used without departing from the scope of the invention. Each host device on the network has at least one IP address that is its own unique identifier and acts as a connectionless protocol. The connection between end points during a communication is not continuous. When a user sends or receives data or messages, the data or messages are divided into components known as packets. Every packet is treated as an independent unit of data and routed to its final destination—but not necessarily via the same path.

### III. Wireless Networks

Wireless networks can incorporate a variety of types of mobile devices, such as, e.g., cellular and wireless telephones, PCs (personal computers), laptop computers, wearable computers, cordless phones, pagers, headsets, printers, PDAs, etc. For example, mobile devices may include digital systems to secure fast wireless transmissions of voice and/or data. Typical mobile devices include some or all of the following components: a transceiver (for example a transmitter and a receiver, including a single chip transceiver with an integrated transmitter, receiver and, if desired, other functions); an antenna; a processor; display; one or more audio transducers (for example, a speaker or a microphone as in devices for audio communications); electromagnetic data storage (such as ROM, RAM, digital data storage, etc., such as in devices where data processing is provided); memory; flash memory; and/or a full chip set or integrated circuit; interfaces (such as universal serial bus (USB), coder-decoder (CODEC), universal asynchronous receiver-transmitter (UART), phase-change memory (PCM), etc.). Other components can be provided without departing from the scope of the invention.

Wireless LANs (WLANs) in which a mobile user can connect to a local area network (LAN) through a wireless connection may be employed for wireless communications. Wireless communications can include communications that propagate via electromagnetic waves, such as light, infrared, radio, and microwave. There are a variety of WLAN standards that currently exist, such as Bluetooth®, IEEE 802.11, and the obsolete HomeRF.

By way of example, Bluetooth products may be used to provide links between mobile computers, mobile phones, portable handheld devices, personal digital assistants (PDAs), and other mobile devices and connectivity to the Internet. Bluetooth is a computing and telecommunications industry specification that details how mobile devices can easily interconnect with each other and with non-mobile devices using a short-range wireless connection. Bluetooth creates a digital wireless protocol to address end-user problems arising from the proliferation of various mobile devices that need to keep data synchronized and consistent from one device to another, thereby allowing equipment from different vendors to work seamlessly together.

An IEEE standard, IEEE 802.11, specifies technologies for wireless LANs and devices. Using 802.11, wireless networking may be accomplished with each single base station supporting several devices. In some examples, devices may come pre-equipped with wireless hardware or a user may install a separate piece of hardware, such as a card, that may include an antenna. By way of example, devices used in 802.11 typically include three notable elements, whether or not the device is an access point (AP), a mobile station (STA), a bridge, a personal computing memory card International Association (PCMCIA) card (or PC card) or another device: a radio transceiver; an antenna; and a MAC (Media Access Control) layer that controls packet flow between points in a network.

In addition, Multiple Interface Devices (MIDs) may be utilized in some wireless networks. MIDs may contain two independent network interfaces, such as a Bluetooth interface and an 802.11 interface, thus allowing the MID to participate on two separate networks as well as to interface with Bluetooth devices. The MID may have an IP address and a common IP (network) name associated with the IP address.

Wireless network devices may include, but are not limited to Bluetooth devices, WiMAX (Worldwide Interoperability for Microwave Access), Multiple Interface Devices (MIDs), 802.11x devices (IEEE 802.11 devices including, 802.11a, 802.11b and 802.11g devices), HomeRF (Home Radio Frequency) devices, Wi-Fi (Wireless Fidelity) devices, GPRS (General Packet Radio Service) devices, 3 G cellular devices, 2.5 G cellular devices, GSM (Global System for Mobile Communications) devices, EDGE (Enhanced Data for GSM Evolution) devices, TDMA type (Time Division Multiple Access) devices, or CDMA type (Code Division Multiple Access) devices, including CDMA2000. Each network device may contain addresses of varying types including but not limited to an IP address, a Bluetooth Device Address, a Bluetooth Common Name, a Bluetooth IP address, a Bluetooth IP Common Name, an 802.11 IP Address, an 802.11 IP common Name, or an IEEE MAC address.

Wireless networks can also involve methods and protocols found in, Mobile IP (Internet Protocol) systems, in PCS systems, and in other mobile network systems. With respect to Mobile IP, this involves a standard communications protocol created by the Internet Engineering Task Force (IETF). With Mobile IP, mobile device users can move across networks while maintaining their IP Address assigned once. See Request for Comments (RFC) 3344. NB: RFCs are formal documents of the Internet Engineering Task Force (IETF). Mobile IP enhances Internet Protocol (IP) and adds a mechanism to forward Internet traffic to mobile devices when connecting outside their home network. Mobile IP assigns each mobile node a home address on its home network and a care-of-address (CoA) that identifies the current location of the device within a network and its subnets. When a device is moved to a different network, it receives a new care-of address. A mobility agent on the home network can associate each home address with its care-of address. The mobile node can send the home agent a binding update each time it changes its care-of address using Internet Control Message Protocol (ICMP).

FIG. 1C depicts components that can be employed in system configurations enabling the systems and technical effect of this disclosure, including wireless access points to which client devices communicate. In this regard, FIG. 1C shows a wireless network 150 connected to a wireless local area network (WLAN) 152. The WLAN 152 includes an access point (AP) 154 and a number of user stations 156, 156′. For example, the network 150 can include the Internet or a corporate data processing network. The access point 154 can be a wireless router, and the user stations 156, 156′ can be portable computers, personal desk-top computers, PDAs, portable voice-over-IP telephones and/or other devices. The access point 154 has a network interface 158 linked to the network 150, and a wireless transceiver in communication with the user stations 156, 156′. For example, the wireless transceiver 160 can include an antenna 162 for radio or microwave frequency communication with the user stations 156, 156′. The access point 154 also has a processor 164, a program memory 166, and a random access memory 168. The user station 156 has a wireless transceiver 170 including an antenna 172 for communication with the access point station 154. In a similar fashion, the user station 156′ has a wireless transceiver 170′ and an antenna 172 for communication to the access point 154. By way of example, in some embodiments an authenticator could be employed within such an access point (AP) and/or a supplicant or peer could be employed within a mobile node or user station. Desktop 108 and key board 118 or input devices can also be provided with the user status.

### IV. Computer Network Environment

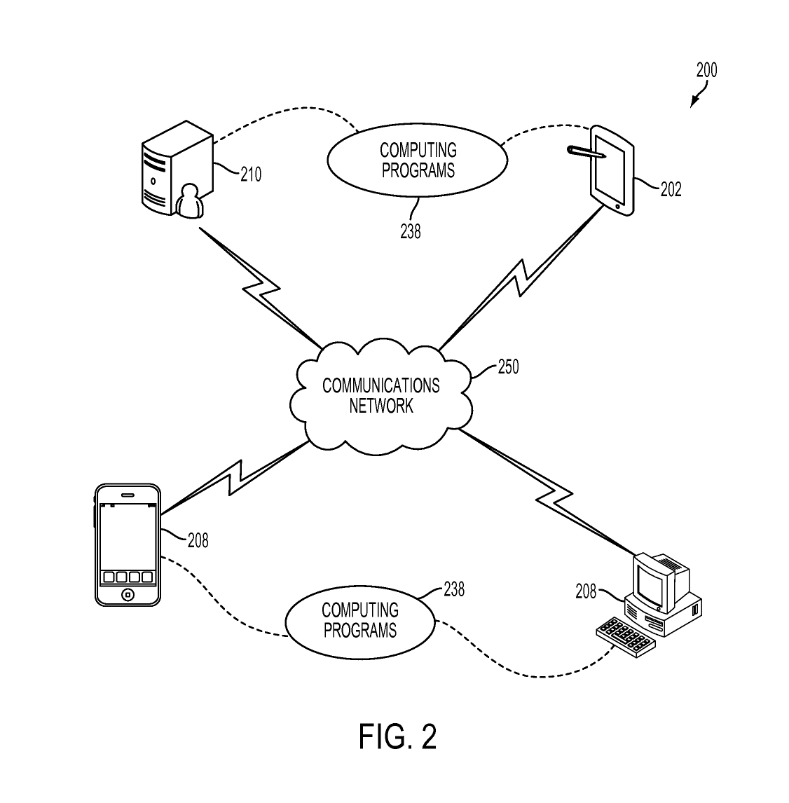

Computing system 100, described above, can be deployed as part of a computer network used to achieve the desired technical effect and transformation. In general, the above description for computing environments applies to both server computers and client computers deployed in a network environment. FIG. 2 illustrates an exemplary illustrative networked computing environment 200, with a server in communication with client computers via a communications network 250. As shown in FIG. 2, server 210 may be interconnected via a communications network 250 (which may be either of, or a combination of a fixed-wire or wireless LAN, WAN, intranet, extranet, peer-to-peer network, virtual private network, the Internet, or other communications network) with a number of client computing environments such as tablet personal computer 202, smart phone 208, personal computer 202, and personal digital assistant. In a network environment in which the communications network 250 is the Internet, for example, server 210 can be dedicated computing environment servers operable to process and communicate data to and from client computing environments via any of a number of known protocols, such as, hypertext transfer protocol (HTTP), file transfer protocol (FTP), simple object access protocol (SOAP), or wireless application protocol (WAP). Other wireless protocols can be used without departing from the scope of the disclosure, including, for example Wireless Markup Language (WML), DoCoMo i-mode (used, for example, in Japan) and XHTML Basic. Additionally, networked computing environment 200 can utilize various data security protocols such as secured socket layer (SSL) or pretty good privacy (PGP). Each client computing environment can be equipped with operating system 238 operable to support one or more computing applications, such as a web browser (not shown), or other graphical user interface (not shown), or a mobile desktop environment (not shown) to gain access to server computing environment 200.

In operation, a user (not shown) may interact with a computing application running on a client computing environment to obtain desired data and/or computing applications. The data and/or computing applications may be stored on server computing environment 200 and communicated to cooperating users through client computing environments over exemplary communications network 250. The computing applications, described in more detail below, are used to achieve the desired technical effect and transformation set forth. A participating user may request access to specific data and applications housed in whole or in part on server computing environment 200. These data may be communicated between client computing environments and server computing environments for processing and storage. Server computing environment 200 may host computing applications, processes and applets for the generation, authentication, encryption, and communication data and applications and may cooperate with other server computing environments (not shown), third party service providers (not shown), network attached storage (NAS) and storage area networks (SAN) to realize application/data transactions.

### V. Devices for Measuring Peak Flow-Rate which are Configurable to Operate in the Computing and Network Environments to Achieve a Desired Technical Effect or Transformation

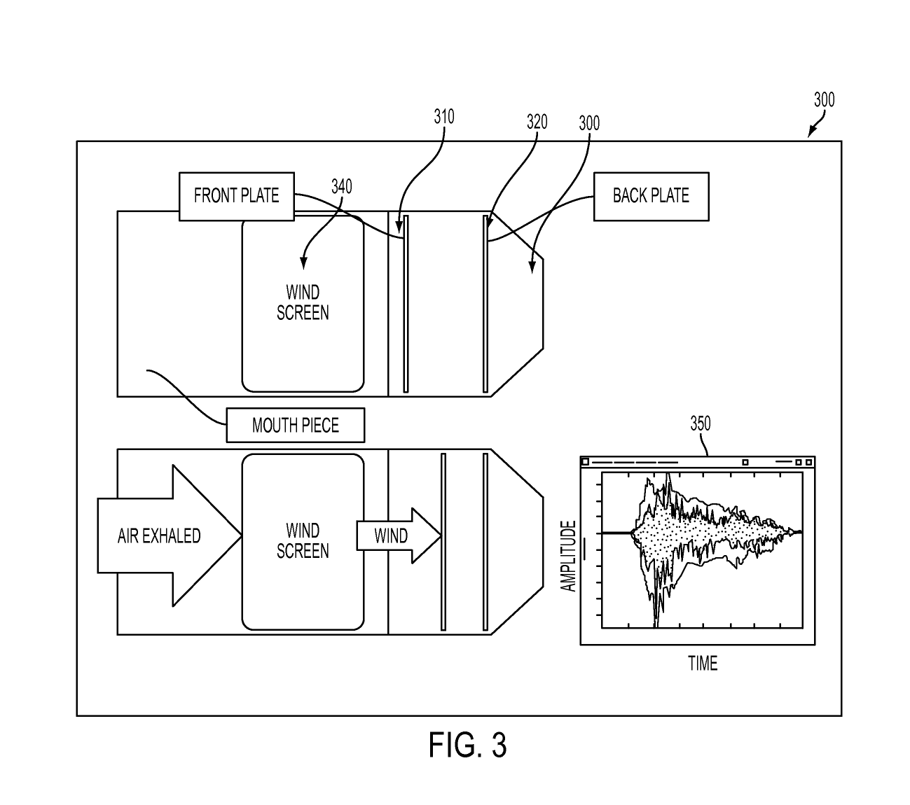

FIG. 3 illustrates an operating principle of a condenser microphone, suitable for use in flow-rate detection devices of the disclosure. A condenser microphone has a front plate 310 (diaphragm), a back plate 320 positioned behind the diaphragm 330. The two plates act as the two plates of a capacitor. An electric charge can be stored between the two plates. When the front plate is vibrated or moved (e.g., with the application of sound waves), the distance between the two plates changes which results in a chance of capacitance. The capacitance of a diaphragm 310—back plate 320 capacitor and the value of a built-in resistor form a filter which is high pass for an audio signal and low pass for a bias voltage. The voltage across the resistor (e.g., the output of the condenser microphone) is then amplifiable for recording. A wind screen 340 can also be provided to reduce the impact of air from secondary sources. The resulting output has the quality shown in the graph 350, where time from 0 to 700 msec is on the x axis and amplitude from −0.5 to 0.3 is on the y axis.

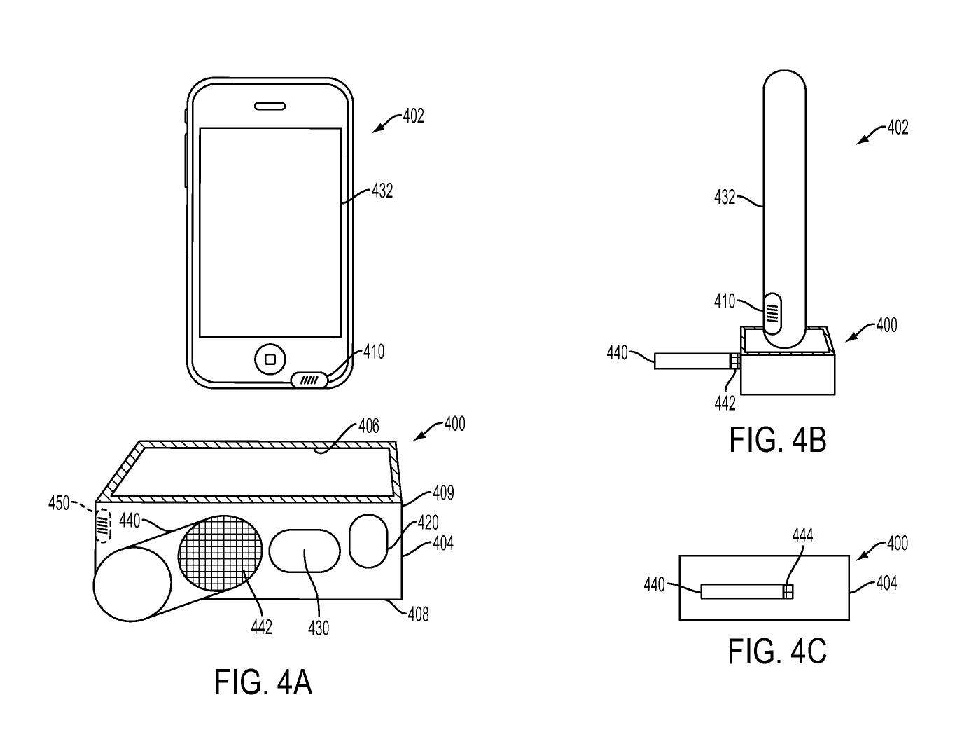

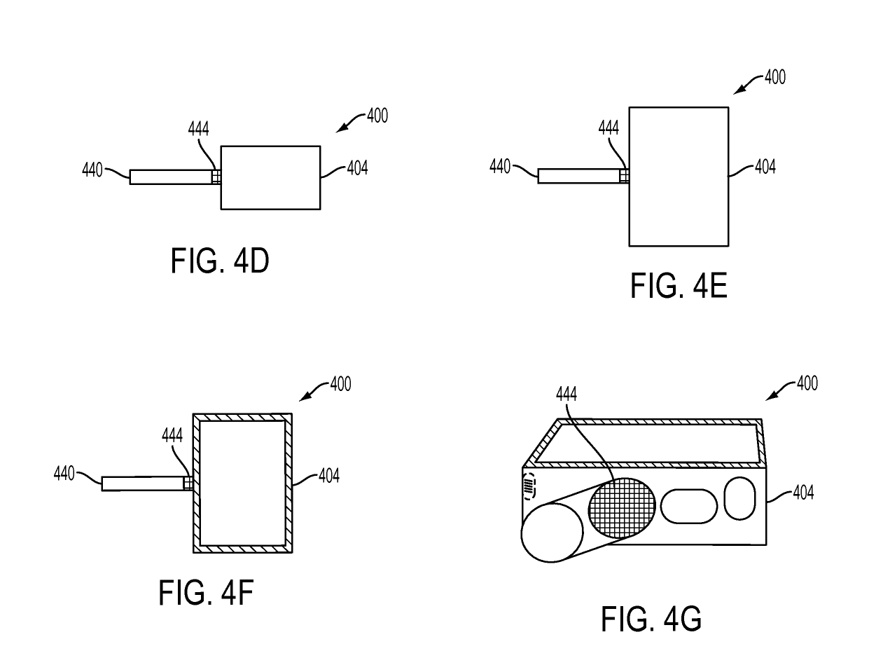

FIGS. 4A-G illustrate a flow-rate detection device 400 configurable in communication with a secondary electronic device 402, such as a smart phone, that provides a microphone, such as ambient microphone 410 shown, a membrane sensor microphone, or any other suitable microphone device capable of capturing sound is provided. FIG. 4A illustrates a flow-rate detection device 400 and electronic device 402 from a front view. FIG. 4B illustrates a flow-rate detection device 400 and electronic device 402 from a side view. FIG. 4C illustrates a flow-rate detection device 400 from a front view with the mouth piece 440 in a second, folded, position. FIG. 4D illustrates a flow-rate detection device 400 from a side view with the mouth piece 440 extended outward and ready for use. FIG. 4E illustrates a flow-rate detection device 400 from a bottom view with the mouth piece 440 extended outward and ready for use. FIG. 4F illustrates a flow-rate detection device 400 from a top view with the mouth piece 440 extended outward and ready for use. FIG. 4F illustrates a flow-rate detection device 400 from an elevated view with the mouth piece 440 extended outward and ready for use.

A processor can be provided on the flow-rate detection device 400 to control, for example, operation of the flow-rate detection device, communication with a secondary electronic device 402, and such other processes as would be desirable.

The flow-rate detection device 400 has a power supply (such as removable power supply 450) which is actuated by an on-off button 420. Power supplies include any suitable power supply including removable power supplies such as Li battery, NiCad battery, etc. An optional visual indicator 430 can be provided on the flow-rate detection device 400, such as an LED display, wherein the visual indicator is configurable to provide a visual indication of status or operation of the flow-rate detection device. In alternative configurations, the flow-rate detection device 400 can communicate a visual indication to the secondary electronic device 402, in addition to providing a visual indication or in lieu of providing a visual indication. In at least some configurations, the flow-rate detection device 400 is adaptable and configurable to transmit a display instruction to the secondary electronic device 402 having a visual indicator or display 432. Where the visual indicator information is transmitted or communicated to the secondary electronic device 402, the secondary electronic device display 432 then displays the visual indication of status or operation.

Additionally or alternatively, the flow-rate detection device 400 can be configured to include an audible indicator which is adapted to provide audible information to a user when the flow-rate detection device is in use. In alternative configurations, the flow-rate detection device 400 can communicate an audible indication to the secondary electronic device 402, in addition to providing an audible indication or in lieu of providing an audible indication. As will be appreciated by those skilled in the art, audible indicators might be particularly useful to visually impaired users. Similarly, tactile displays can also be provided.

The flow-rate detection device is adaptable and configurable to communicate with an electronic device, such as a cell phone or smart phone, which has communication functionality. As shown in FIGS. 4A-G, the flow-rate detection device 400 has a housing 404 which is configurable to, for example, surround a portion of the secondary electronic device 402. As illustrated, the housing 404 has a lower surface 408, side walls 409 and defines an opening configured to receive the secondary electronic device 402 wherein the opening has an interior wall 406. The form factor of the housing as been illustrated as rectangular for ease of illustration, but, as will be appreciated by those skilled in the art, the form factor of a particular housing will be optimized for interaction and/or communication with the form factor of a secondary electronic device 402 (e.g., Apple® iPhone, RIM Blackberry®, etc.) and a wide variety of form factors and cross-sectional shapes can be used (e.g., square, rectangular, oval, etc.) without departing from the scope of the disclosure. Additionally, although not depicted, the housing of any of the embodiments that engage a secondary electronic device 402 can be configured to abut, without enclosing, the form factor of the secondary electronic device. Alternatively, the housing can engage the rear surface of the secondary electronic device, enclose a bottom portion of the electronic device, or engage the front surface of the secondary electronic device.

In other configurations, the housing 404 can be configured to removeably engage the secondary electronic device in a position proximate to or adjacent to the speaker 410 or the camera. The housing 404 can further be configurable to engage a wide variety of secondary electronic device configurations.

The indicators provide information to a user about operational status and can, in some configurations, be used to communicate with a user to improve user interaction with the flow-rate detection device.

A mouth piece 440 can also be provided, as shown in FIGS. 4A-B. The flow-rate detection device is in communication with an electronic device 402 having a microphone 410. The flow-rate detection device has a mouth piece 440 is also configurable such that a filter 442 can be positioned between the mouth piece and the microphone 410 of the secondary electronic device. For example, as illustrated, the microphone is positioned on an upper surface of the secondary electronic device, and the mouth piece is positioned such that inspiration or expiration by a patient on the mouth piece will result in the sound be communicated to the microphone du to the proximity of the mouthpiece to the microphone. As will be appreciated any suitable material can be used as a filter. Typically the filter is configurable to prevent particulate matter, e.g. pollen, from entering into the respiratory system during use. The flow-rate detection device includes a power supply, such as removable power supply 450.

The mouth piece can be integrally formed such that it is formed from one piece or formed such that it has unitary operation when formed. In at least some configurations, the mouthpiece can be hinged 444 or bendable such that when the flow-rate detection device 400 is not connected to or in communication with the secondary electronic device, the mouth piece is rotatable along a hinged section to achieve a lower profile flow-rate detection device as shown in the side view of the flow-rate detection device depicted in FIG. 4c.

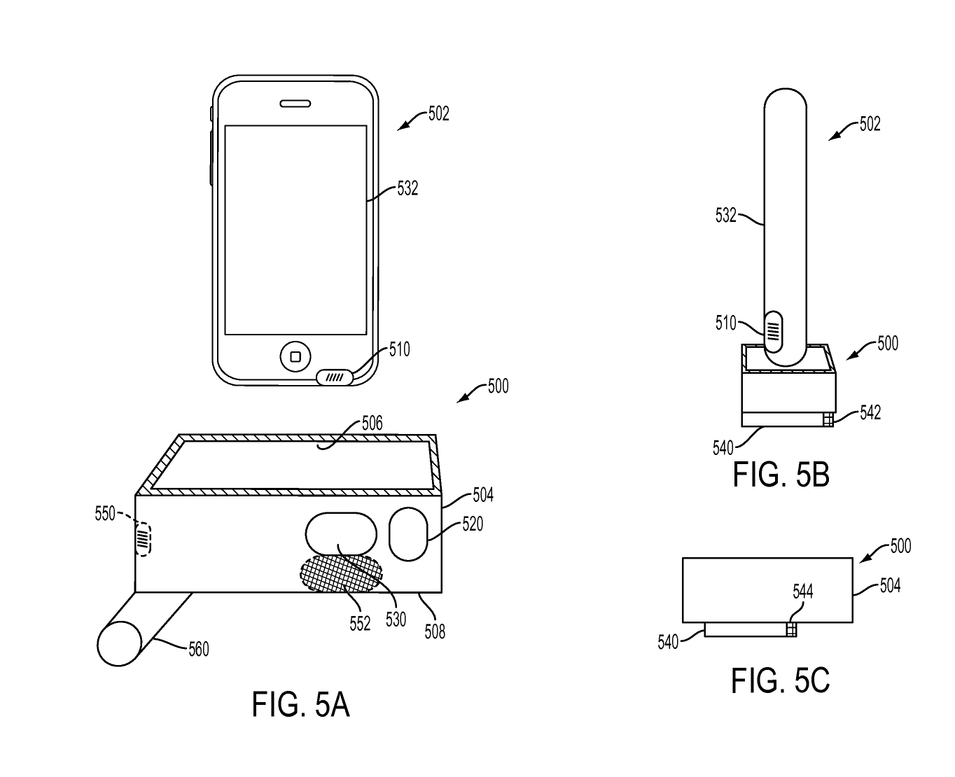

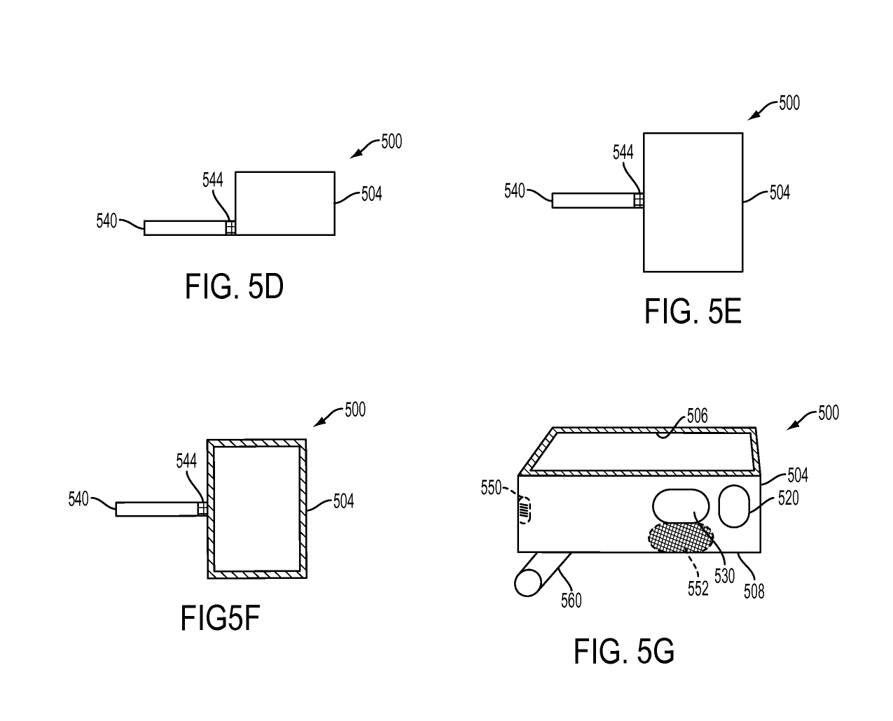

As shown in FIGS. 5A-G the mouthpiece 540 can be configured on the bottom of the flow-rate detection device 500. FIG. 5A illustrates a flow-rate detection device 500 and electronic device 502 from a front view. FIG. 5B illustrates a flow-rate detection device 500 and electronic device 502 from a side view. FIG. 5c illustrates a flow-rate detection device 500 from a front view with the mouth piece 540 in a second, folded, position. FIG. 5D illustrates a flow-rate detection device 500 from a side view with the mouth piece 540 extended outward and ready for use. FIG. 5E illustrates a flow-rate detection device 500 from a bottom view with the mouth piece 540 extended outward and ready for use. FIG. 5F illustrates a flow-rate detection device 500 from a top view with the mouth piece 540 extended outward and ready for use. FIG. 5G illustrates a flow-rate detection device 500 from an elevated view with the mouth piece 540 extended outward and ready for use.

The flow-rate detection device 500 has a housing 504 which is configurable to, for example, surround a portion of the secondary electronic device 502. As illustrated, the housing 504 has a lower surface 508, side walls 509 and defines an opening configured to receive the secondary electronic device wherein the opening has an interior wall 506. As with other configurations, the housing 504 can be configured to removeably engage the secondary electronic device in a position proximate to or adjacent to the speaker 510. As illustrated in this configuration, the speaker is positioned on the bottom side of the secondary electronic device. The housing 504 can further be configurable to engage a wide variety of secondary electronic device configurations. Additionally, an on-off button 520, an optional visual indicator 530, and a power supply 550 is provided. The mouth piece 540 is positioned on the lower surface 508 of the housing 504. The mouth piece can be removeable and/or hinged to allow the mouth piece to be positioned flat, or substantially flat, against the housing 504 when not in use or when in a storage condition. The mouth piece 540 can be extended from the housing or rotated away from the housing to provide clearance off the end of the mouth piece away from the housing.

As will be appreciated by those skilled in the art, since condenser microphones are relying on the force of sound wave to vibrate the front-plate diaphragm to pick up the sound, it is possible for wind (natural wind or human generated vocal plosives) to push the diaphragm unintentionally. In a worst case scenario, the front diaphragm plate can be pushed by strong wind such that it translates into the back plate. As a result, the diaphragm cannot vibrate anymore and no audio signal is obtained from the microphone. This phenomenon is called plosive or pop.

The mouth piece can further be configurable to provide a removable and/or disposable unit which forms part of a kit. An integrally formed or removeable wind screen 560 can also be provided as shown in FIG. 5. Alternatively, the windscreen can also be removable and/or disposable as another kit component.

As further illustrated in FIG. 5, the flow-rate detection device is configurable to engage a portable electronic device such as a smart phone 208. The flow-rate detection device can be connected to or engage the portable electronic device or can communicate with the portable electronic device wirelessly. The flow-rate detection device in combination with the portable electronic device can further be configured or programmed to perform vibration analysis of the microphone diaphragm of the probe and display the result—either on the flow-rate detection device, on the portable electronic device, or on a computing device in communication with the flow-rate detection device via a communication network.

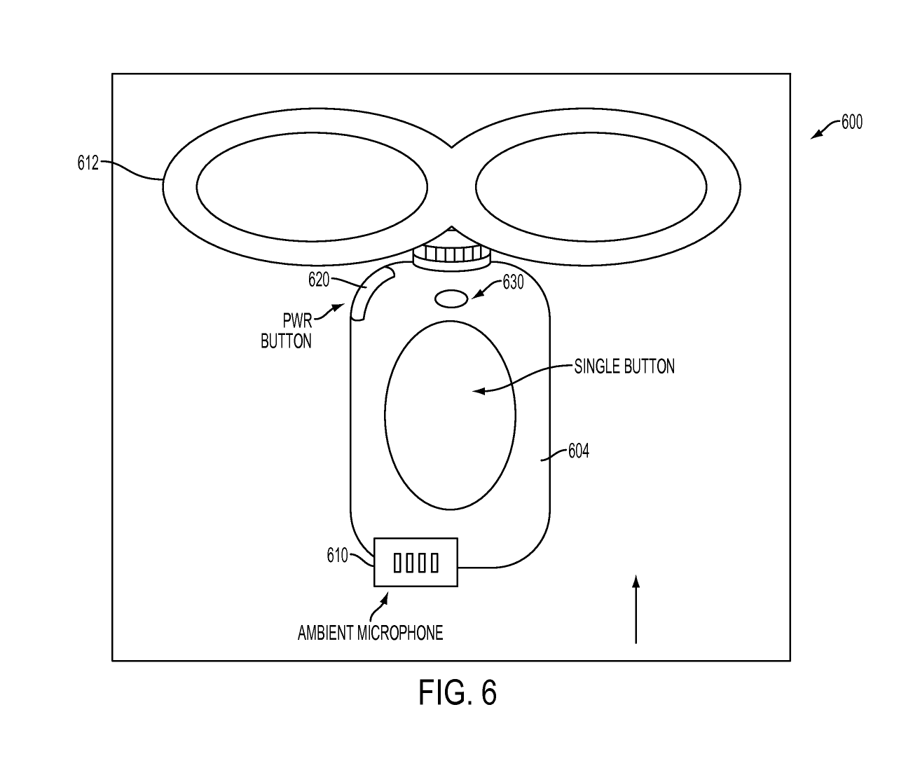

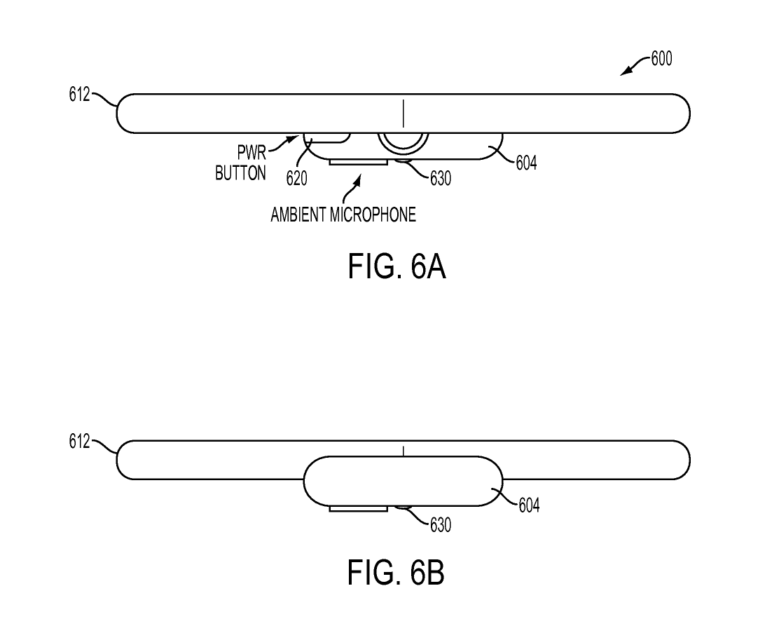

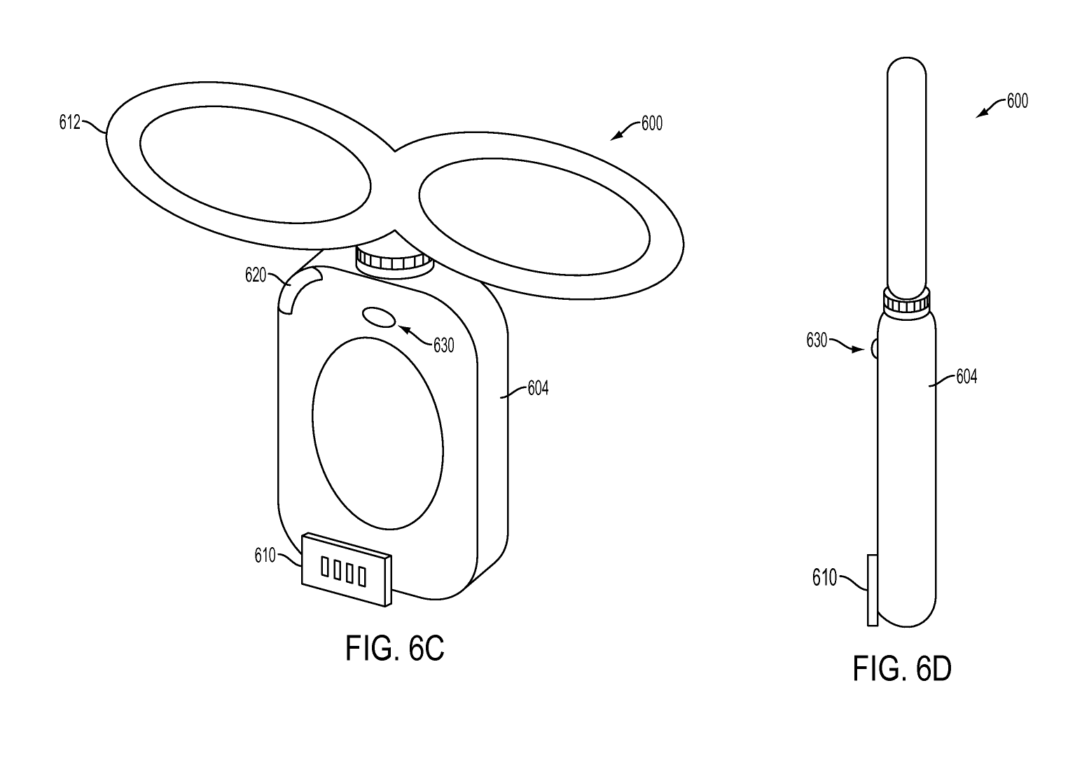

FIGS. 6-6D illustrates an alternative embodiment of a flow-rate detection device 600 adapted and configured to measure inspiratory and/or expiratory flow from a patient wherein a microphone 610 is provided on the flow-rate detection device housing 604. A power button 620 and visual indicator 630 can also be provided. As illustrated here acoustic sensors 612 can be provided in lieu of a mouth piece (as illustrated above). The flow-rate detection device 600 is configurable to communicate a detected respiration related value to a secondary electronic device, such as a cell phone. Alternatively, the flow-rate detection device can be configured to analyze the respiration related value and then to communicate the analyzed information to the secondary electronic device.

Additionally, the data from the diaphragm can be transmitted via a network to a central location where the vibration analysis of the microphone diaphragm of the probe can be performed, the results can then be transmitted back over the network to the user. In at least some configurations, the network can collect information regarding one or more readings from a patient and transmit that information to another location (e.g., to a healthcare provider, or to an emergency service if the readings indicate a dangerous reading), or can be made available to a user in a data management system.

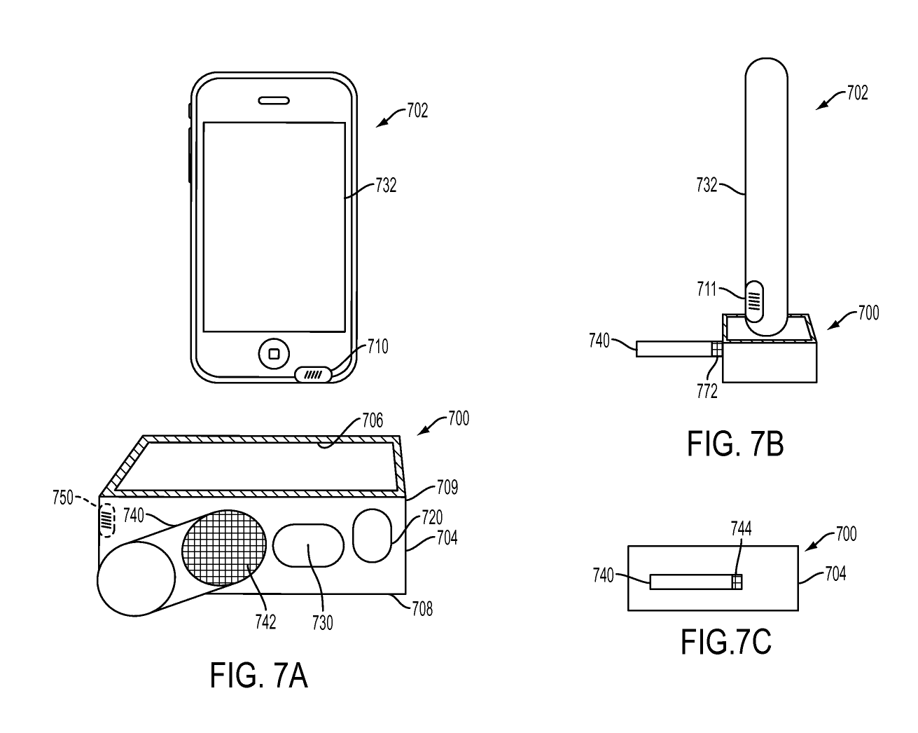

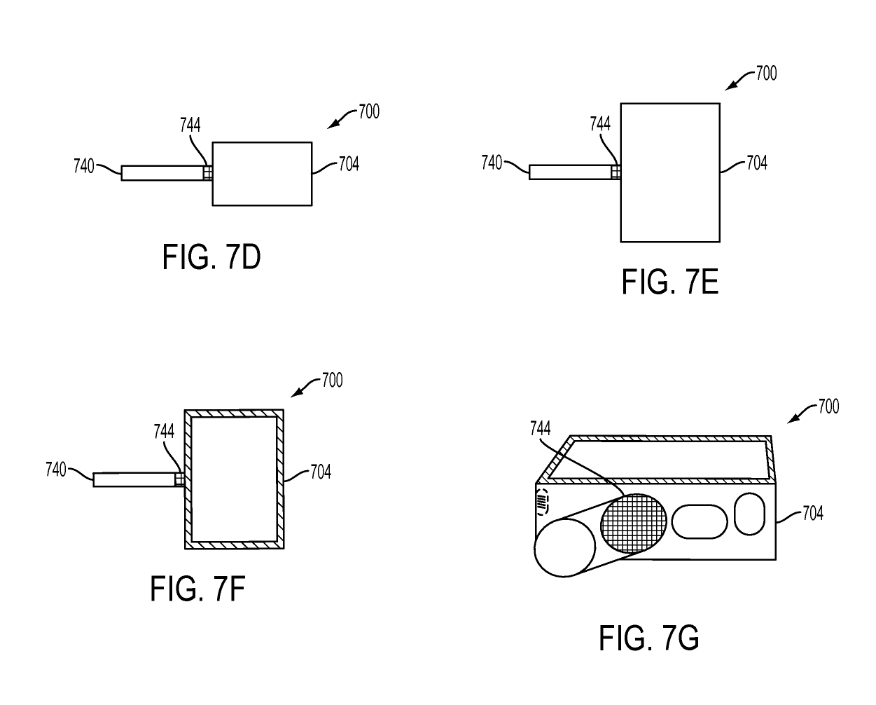

FIGS. 7A-G illustrate a flow-rate detection device 700 configurable in communication with a secondary electronic device 702 that provides a camera 711 or any other suitable device capable of capturing an image. FIG. 7A illustrates a flow-rate detection device 700 and electronic device 702 from a front view. FIG. 7B illustrates a flow-rate detection device 700 and electronic device 702 from a side view. FIG. 7c illustrates a flow-rate detection device 700 from a front view with the mouth piece 740 in a second, folded, position. FIG. 7D illustrates a flow-rate detection device 700 from a side view with the mouth piece 740 extended outward and ready for use. FIG. 7E illustrates a flow-rate detection device 700 from a bottom view with the mouth piece 740 extended outward and ready for use. FIG. 7F illustrates a flow-rate detection device 700 from a top view with the mouth piece 740 extended outward and ready for use. FIG. 7E illustrates a flow-rate detection device 700 from an elevated view with the mouth piece 740 extended outward and ready for use.

A processor can be provided on the flow-rate detection device 700 to control, for example, operation of the flow-rate detection device, communication with a secondary electronic device 702, and such other processes as would be desirable.

The flow-rate detection device 700 has a power supply (such as removable power supply 750) which is actuated by an on-off button 720. Power supplies include any suitable power supply including removable power supplies such as Li battery, NiCad battery, etc. An optional visual indicator 730 can be provided on the flow-rate detection device 700, such as an LED display, wherein the visual indicator is configurable to provide a visual indication of status or operation of the flow-rate detection device. In at least some configurations, the flow-rate detection device 700 is adaptable and configurable to transmit a display instruction to the secondary electronic device having a visual indicator or display 732. Where the visual indicator information is transmitted or communicated to the secondary electronic device, the secondary electronic device display 732 then displays the visual indication of status or operation. Additionally or alternatively, an audible indicator can also be provided which provides audible information to a user when the flow-rate detection device is in use. Audible indicators might be particularly useful to visually impaired users. Similarly, tactile displays can also be provided.

The flow-rate detection device is adaptable and configurable to communicate with an electronic device, such as a cell phone or smart phone, which has communication functionality. As shown in FIGS. 7A-G, the flow-rate detection device 700 has a housing 704 which is configurable to, for example, surround a portion of the secondary electronic device 702. As illustrated, the housing 704 has a lower surface 708, side walls 709 and defines an opening configured to receive the secondary electronic device wherein the opening has an interior wall 706. The form factor of the housing as been illustrated as rectangular for ease of illustration, but, as will be appreciated by those skilled in the art, the form factor of a particular housing will be optimized for interaction with the form factor of a secondary electronic device (e.g., Apple® iPhone, RIM Blackberry®, etc.) and a wide variety of form factors and cross-sectional shapes can be used (e.g., square, rectangular, oval, etc.) without departing from the scope of the disclosure. Additionally, although not depicted, the housing of any of the embodiments that engage a secondary electronic device can be configured to abut, without enclosing, the form factor of the secondary electronic device.

In other configurations, the housing 704 can be configured to removeably engage the secondary electronic device 702 in a position proximate to or adjacent to a camera 711 such that the camera can, for example, count the number, duration, and quality of breaths a user blows into a mouth piece of the flow-rate detection device 700. The housing 704 can further be configurable to engage a wide variety of secondary electronic device configurations taking into consideration, for example, positioning of a camera.

The indicators provide information to a user about operational status and can, in some configurations, be used to communicate with a user to improve user interaction with the flow-rate detection device.

A mouth piece 740 can also be provided, as shown in FIGS. 7A-B. The flow-rate detection device is in communication with an electronic device 702 having a camera 710. The flow-rate detection device has a mouth piece 740 is also configurable such that a filter 742 can be positioned between the mouth piece and the microphone 710 of the secondary electronic device. For example, as illustrated, the microphone is positioned on an upper surface of the secondary electronic device, and the mouth piece is positioned such that inspiration or expiration by a patient on the mouth piece will result in the sound be communicated to the microphone du to the proximity of the mouthpiece to the microphone. As will be appreciated any suitable material can be used as a filter. Typically the filter is configurable to prevent particulate matter, e.g. pollen, from entering into the respiratory system during use. The flow-rate detection device includes a power supply, such as removable power supply 750.

The mouth piece can be integrally formed such that it is formed from one piece or formed such that it has unitary operation when formed. In at least some configurations, the mouthpiece can be hinged 744 or bendable such that when the flow-rate detection device 700 is not connected to or in communication with the secondary electronic device, the mouth piece is rotatable along a hinged section to achieve a lower profile flow-rate detection device as shown in the side view of the flow-rate detection device depicted in FIG. 7c. The assessment made by the camera can be used by any suitable technique including.

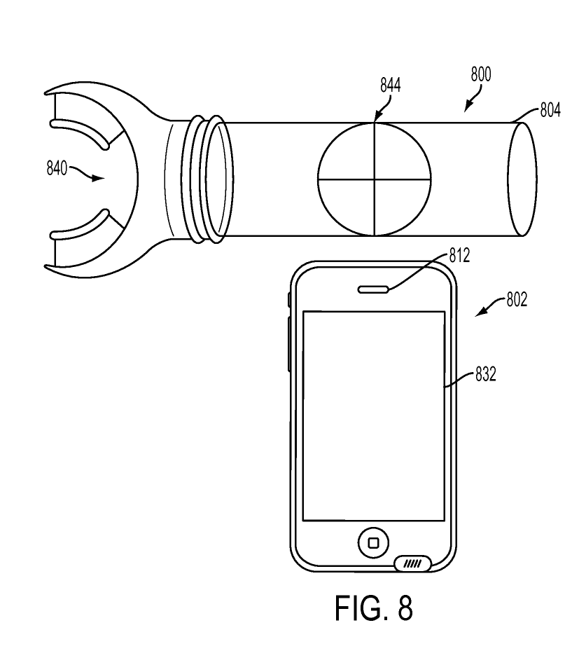

Turning to FIG. 8, a device 800 is depicted which has a mouth piece 840 configurable to fit within the mouth of a patient and includes bite wings 842. The device 800 is adaptable and configurable to position a spinning vein or wheel 844 adjacent a secondary electronic device camera 812. The device 800 has a housing 804 that attaches, for example, to the phone 802 allowing the camera to view proximally the rotation of a turbine vein 844 that is connected within the housing 804 of the device. The spinning vein or wheel 844 is constructed to allow the wheel or vein to rotate freely when air passes across an axis of the vein. The connection between the spinning wheel and housing can be by an axis or the wheel or vein itself can have integral to it a fulcrum or stem allowing it to be inserted into the housing again allowing free rotation of the wheel or vein. The housing 804 would then have two openings at each end and the housing would essentially look like a cylinder allowing for the persons mouth to attach to one end of the housing and the other end of the housing would remain open allowing the individual to freely breathe through the housing. As the person breathes the wheel or vein contained within the housing will spin and the camera from the phone will count the number of rotations. Those rotations are then correlated by the phone or processing device to provide an accurate measurement of the persons breathing flows and then calculate spirometery. The flow is then integrated over time to provide a calculation of volume. The direction of the wheel spinning will indicate the direction of the breath—inspiratory or expiratory breathing. The housing can have as an option a mouthpiece to improve the seal of the persons mouth to the housing/wheel assembly. The camera function can operate in either a series of photo shots or frames taken or a video sequence whereby a delineated number of frames per second can be optimized to measure the rotation numbers.

Other aspects include one or more networked devices. The networked devices comprise: a memory; a processor; a communicator; a display; and an apparatus for detecting expiry flow-rate as discussed herein.

In some aspects communication systems are provided. The communication systems comprise: an apparatus for detecting expiry flow-rate as described herein; a server computer system; a measurement module on the server computer system for permitting the transmission of a flow-rate measurement from the device for measuring the characteristic of the flow-rate over a network; at least one of an API engine connected to at least one of the system for measuring the characteristic of the flow-rate to create a message about the flow-rate measurement and transmit the message over an API integrated network to a recipient having a predetermined recipient user name, an SMS engine connected to at least one of the system for measuring the characteristic of the flow-rate to create an SMS message about the flow-rate measurement and transmit the SMS message over a network to a recipient device having a predetermined flow-rate measurement recipient telephone number, and an email engine connected to at least one of the system for measuring the characteristic of the flow-rate to create an email message about the flow-rate measurement and transmit the email message over the network to a flow-rate measurement recipient email having a predetermined flow-rate measurement recipient email address. A storing module can also be provided on the server computer system for storing the flow-rate measurement on the system for measuring the characteristic of the flow-rate server database. Moreover, at least one of the system for measuring the characteristic of the flow-rate is connectable to the server computer system over at least one of a mobile phone network and an Internet network, and a browser on the flow-rate measurement recipient electronic device is used to retrieve an interface on the server computer system. Additionally, a plurality of email addresses are held in a system for measuring the characteristic of the flow-rate database and fewer than all the email addresses are individually selectable from the computer system, the email message being transmitted to at least one flow-rate measurement recipient email having at least one selected email address. In some instances at least one of the system for measuring the characteristic of the flow-rate is connectable to the server computer system over the Internet, and a browser on the flow-rate measurement recipient electronic device is used to retrieve an interface on the server computer system. Where the system is in communication with, for example, a healthcare provider a plurality of user names are held in the system for detecting expiry flow-rates database and fewer than all the user names are individually selectable from the computer system, the message being transmitted to at least one flow-rate measurement recipient user name via an API. The flow-rate measurement recipient electronic device can also be connectable to the server computer system over the Internet, and a browser on the flow-rate measurement recipient electronic device is used to retrieve an interface on the server computer system. The flow-rate measurement recipient electronic device may also be connected to the server computer system over a cellular phone network, such as where the electronic device is a mobile device. Additionally, the system can include an interface on the server computer system, the interface being retrievable by an application on the flow-rate measurement recipient mobile device. In some cases, the SMS flow-rate measurement is received by a message application on the flow-rate measurement recipient mobile device. Where a plurality of SMS flow-rate measurements are received for the flow-rate measurement, each by a respective message application on a respective flow-rate measurement recipient mobile device. At least one SMS engine can be configured to receive an SMS response over the cellular phone SMS network from the flow-rate measurement recipient mobile device and stores an SMS response on the server computer system. Additionally, a flow-rate measurement recipient phone number ID is transmitted with the SMS flow-rate measurement to the SMS engine and is used by the server computer system to associate the SMS flow-rate measurement with the SMS response. Moreover, the server computer system can be connectable over a cellular phone network to receive a response from the flow-rate measurement recipient mobile device. The SMS flow-rate measurement can also include a URL that is selectable at the flow-rate measurement recipient mobile device to respond from the flow-rate measurement recipient mobile device to the server computer system, the server computer system utilizing the URL to associate the response with the SMS flow-rate measurement. The communication system can further comprise in at least some configurations: a downloadable application residing on the flow-rate measurement recipient mobile device, the downloadable application transmitting the response and a flow-rate measurement recipient phone number ID over the cellular phone network to the server computer system, the server computer system utilizing the flow-rate measurement recipient phone number ID to associate the response with the SMS flow-rate measurement. In other configurations, the system can comprise: a transmissions module that transmits the flow-rate measurement over a network other than the cellular phone SMS network to a flow-rate measurement recipient user computer system, in parallel with the flow-rate measurement that is sent over the cellular phone SMS network, and/or a downloadable application residing on the flow-rate measurement recipient host computer, the downloadable application transmitting a response and a flow-rate measurement recipient phone number ID over the cellular phone network to the server computer system, the server computer system utilizing the flow-rate measurement recipient phone number ID to associate the response with the SMS flow-rate measurement.

Other aspects include one or more networked apparatuses. The networked apparatuses comprise: a memory; a processor; a communicator; a display; and an apparatus for detecting the expiry flow-rates as described herein.

In some aspects the communication systems comprise: an apparatus for detecting the expiry flow-rates as described herein; a server computer system; a measurement module on the server computer system for permitting the transmission of a flow-rate measurement from the system for measuring the characteristic of the flow-rate over a network; at least one of an API engine connected to at least one of the system for measuring the characteristic of the flow-rate to create a message about the flow-rate measurement and transmit the message over an API integrated network to a recipient having a predetermined recipient user name, an SMS engine connected to at least one of the system for measuring the characteristic of the flow-rate to create an SMS message about the flow-rate measurement and transmit the SMS message over a network to a recipient device having a predetermined flow-rate measurement recipient telephone number, and an email engine connected to at least one of the system for measuring the characteristic of the flow-rate to create an email message about the flow-rate measurement and transmit the email message over the network to a flow-rate measurement recipient email having a predetermined flow-rate measurement recipient email address. A storing module can also be provided on the server computer system for storing the flow-rate measurement on the system for measuring the characteristic of the flow-rate server database. Moreover, at least one of the system for measuring the characteristic of the flow-rate is connectable to the server computer system over at least one of a mobile phone network and an Internet network, and a browser on the flow-rate measurement recipient electronic device is used to retrieve an interface on the server computer system. Additionally, a plurality of email addresses are held in a system for measuring the characteristic of the flow-rate database and fewer than all the email addresses are individually selectable from the computer system, the email message being transmitted to at least one flow-rate measurement recipient email having at least one selected email address. In some instances at least one of the system for measuring the characteristic of the flow-rate is connectable to the server computer system over the Internet, and a browser on the flow-rate measurement recipient electronic device is used to retrieve an interface on the server computer system. Where the system is in communication with, for example, a healthcare provider a plurality of user names are held in the system for detecting expiry flow-rates database and fewer than all the user names are individually selectable from the computer system, the message being transmitted to at least one flow-rate measurement recipient user name via an API. The flow-rate measurement recipient electronic device can also be connectable to the server computer system over the Internet, and a browser on the flow-rate measurement recipient electronic device is used to retrieve an interface on the server computer system. The flow-rate measurement recipient electronic device may also be connected to the server computer system over a cellular phone network, such as where the electronic device is a mobile device. Additionally, the system can include an interface on the server computer system, the interface being retrievable by an application on the flow-rate measurement recipient mobile device. In some cases, the SMS flow-rate measurement is received by a message application on the flow-rate measurement recipient mobile device. Where a plurality of SMS flow-rate measurements are received for the flow-rate measurement, each by a respective message application on a respective flow-rate measurement recipient mobile device. At least one SMS engine can be configured to receive an SMS response over the cellular phone SMS network from the flow-rate measurement recipient mobile device and stores an SMS response on the server computer system. Additionally, a flow-rate measurement recipient phone number ID is transmitted with the SMS flow-rate measurement to the SMS engine and is used by the server computer system to associate the SMS flow-rate measurement with the SMS response. Moreover, the server computer system can be connectable over a cellular phone network to receive a response from the flow-rate measurement recipient mobile device. The SMS flow-rate measurement can also include a URL that is selectable at the flow-rate measurement recipient mobile device to respond from the flow-rate measurement recipient mobile device to the server computer system, the server computer system utilizing the URL to associate the response with the SMS flow-rate measurement. The communication system can further comprise in at least some configurations: a downloadable application residing on the flow-rate measurement recipient mobile device, the downloadable application transmitting the response and a flow-rate measurement recipient phone number ID over the cellular phone network to the server computer system, the server computer system utilizing the flow-rate measurement recipient phone number ID to associate the response with the SMS flow-rate measurement. In other configurations, the system can comprise: a transmissions module that transmits the flow-rate measurement over a network other than the cellular phone SMS network to a flow-rate measurement recipient user computer system, in parallel with the flow-rate measurement that is sent over the cellular phone SMS network, and/or a downloadable application residing on the flow-rate measurement recipient host computer, the downloadable application transmitting a response and a flow-rate measurement recipient phone number ID over the cellular phone network to the server computer system, the server computer system utilizing the flow-rate measurement recipient phone number ID to associate the response with the SMS flow-rate measurement.

### VI. Kits

Bundling all devices, tools, components, materials, and accessories needed to use a device to test expiry flow-rate into a kit may enhance the usability and convenience of the devices. Suitable kits, can also include, for example, an electronic expiry flow measurement device, filters, wind screens, electronic device connector or adapter, mouth pieces, filters, power supplies, software programs (apps) configurable to collect information from the devices and/or provide information to a central database or system, alcohol swabs, and the like.

### VII. Systems Configurable to Operate in Computing and Network Environments to Achieve a Desired Technical Effect or Transformation

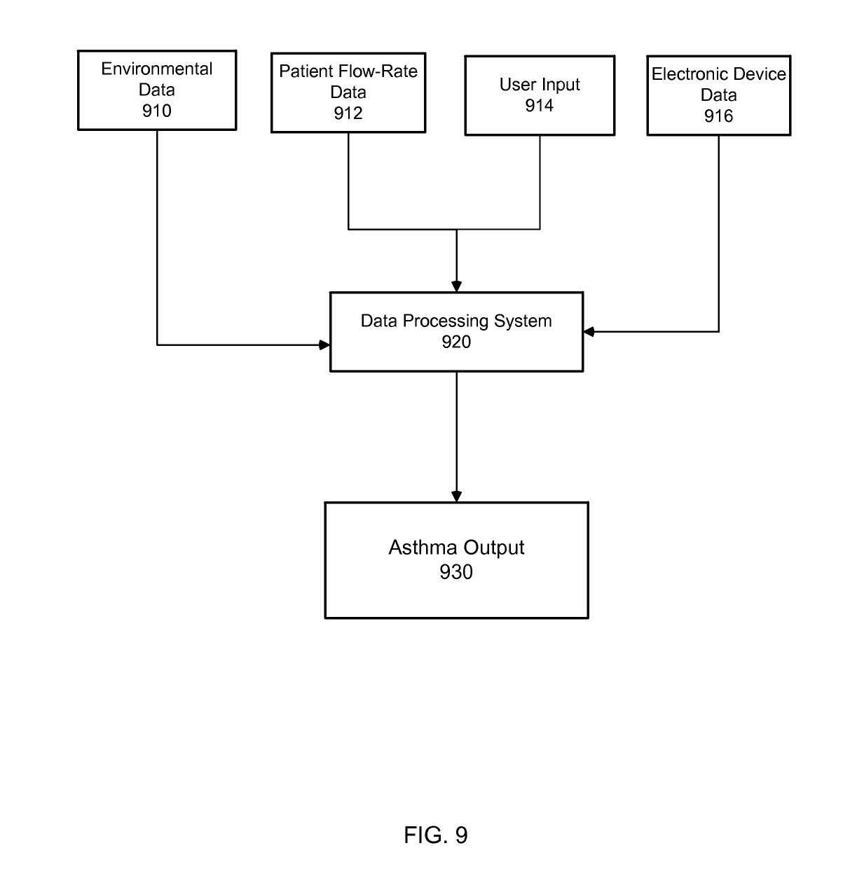

FIG. 9 illustrates the interrelationship between components of a suitable system according to the disclosure. Environmental data 910 can be obtained from a sensor associated with an electronic device, such as those disclosed above in reference to FIG. 2, or can be acquired from a remote source such as a website that provides environmental data based on a location for the electronic device, such as that determined by GPS. Patient flow-rate data 912 is also provided by the flow-rate detector. Additionally, user input 914 can be obtained, if desired, as well as electronic device data 916, such as location, altitude, temperature, etc. The information is then processed, using a data processing system 920 which is located either on a network or on the electronic device, to generate an asthma output 930. The information can then be transmitted back to one or more remote location (such as a physician's office or other users). Additionally, input can be active or passive input. Active input occurs when the user enters information concerning, for example, current conditions, medications, etc. Passive input occurs when the system logs that the user took an action, such as using the device.

The system can also receive data from a secondary measurement device, such as data from a heart rate monitor, a heart sound sensor, and a pulse oximetry device (which senses saturation of oxygen in arterial blood flow). Additionally, the system can also receive behavioral data, such as one or more of data concerning compliance with a medication protocol prescribed by a healthcare provider, compliance with a testing and/or monitoring protocol, compliance with system generated recommendations, compliance with health care provider recommendations, etc.

The system can analyze the environmental information for a particular patient based on one or more of the following: the patient's prior history under similar conditions, the real time results of other system users having a similar profile or a similar history, and the historical results of other system users having a similar profile or a similar history under similar conditions previously experienced.

In at least some configurations, the system dynamically analyzes environmental information based on one or more of the following: the patient's prior history under similar conditions, the real time results of other system users having a similar profile or a similar history, and the historical results of other system users having a similar profile or a similar history under similar conditions previously experienced. Dynamic analysis or processing can be impacted by the passage of time and/or the presence or absence of a power source. In at least some configurations, data is refreshed and/or analyzed at a time interval determined by the system or selected by the user. In some configurations, analysis and processing may occur at a greater rate, e.g. where a detection of travel is sensed or at a smaller rate, where there is no significant movement detected.

The system is configurable to send a patient an alert to the potential of a respiratory episode, suggestions for preparing for a change in environment, historical information about reactions to current or predicted future conditions in a specific geography, and so on. Additionally, the system can provide additional data, alerts, or reports to the user's healthcare provider to enable to healthcare provider to monitor conditions and propose changes in treatment protocol, if desired. In some configurations, the system is configurable to alert emergency services to the location of the user and the nature of the respiratory event.

In some configurations the environmental data can be continuously received during operation of an onboard environmental sensor. Environmental sensor data can, for example, be collected by sensors on or near the body of the patient which may include a humidity sensor, a temperature sensor, an altitude sensor, a GPS sensor, and an airborne particle sensor, or other suitable sensor. The sensor can be associated with the device or with an electronic device. In some instances, environmental data can be pre-processed to generate an indication of environmental asthma triggers. The information can also be compared to historical data for that patient. In other configurations, the environmental information is available from an external source such as www.pollen.com. In some configurations, altitude is determinable based on the GPS location. Other environmental data can be reviewed including, for example, air pollution data, airborne particulate matter date, airborne irritant data, ambient temperature, temperature changes, and humidity data.

Other aspects include one or more networked devices. The networked devices comprise: a memory; a processor; a communicator; a display; and an apparatus for detecting expiry flow-rate as discussed herein.

Communication systems are configurable to have at least one of an API engine connected to at least one of the electronic device to create a message about respiratory episode data and transmit the message over an API integrated network to a recipient having a predetermined recipient user name, an SMS engine connected to the system to create an SMS message about the respiratory episode data and transmit the SMS message over a network to a recipient device having a predetermined respiratory episode data recipient telephone number, and an email engine connected to the system to create an email message about the respiratory episode data and transmit the email message over the network to a recipient email.

A storing module can also be provided on the server computer system for storing the respiratory episode data on the system for measuring the characteristic of the flow-rate server database. Moreover, the system is connectable to a server computer system over at least one of a mobile phone network and an Internet network, and a browser on the recipient electronic device which can be used to retrieve an interface on the server computer system. Additionally, a plurality of email addresses are held in a system database and fewer than all the email addresses are individually selectable from the computer system, the email message being transmitted to at least one data recipient email having at least one selected email address. In some instances the system is connectable to the server computer system over the Internet, and a browser on the electronic device to retrieve an interface on the server computer system. A plurality of user names are held in the system database and fewer than all the user names are individually selectable from the computer system, enabling a message to be transmitted to at least one respiratory episode data recipient user name via an API.

Other aspects include one or more networked apparatuses. The networked apparatuses comprise: a memory; a processor; a communicator; a display; and an apparatus for receiving user input rates as described herein.

### VIII. EXAMPLES

### Example 1

A first user uses a peak-flow rate device which obtains flow-rate data from that user and stores the information on the handheld device. Additionally, the user can also enter additional data which may be relevant or desirable to record at the time of taking the peak-flow measurement into a program on the handheld device, including, for example, how the user is feeling, whether the user is stressed (and the level of stress), whether the user has a headache, etc. This input of additional data can, for example, be achieved by providing for periodic question(s) that display at various times throughout the day or via a more comprehensive patient data input and/or query process. Information can also include geographic positioning data, such as a GPS tag, date and time information, as well as ambient conditions data. Ambient condition data includes, but is not limited to, weather conditions, temperature, pollution, pollen count, air quality, etc. The status of the first user is uploaded to a server via a network. One or more of a second user or subsequent user, who has not necessarily provided peak-flow data, reports that there has been a condition change and the one or more of a second or subsequent user report (or reports) is uploaded to a server via a network. An assessment is then made that both the first and second users are located within a geographic area set by the system for that area (e.g., 0.5 mile radius, 1 mile radius, 1.5 mile radius, 2 mile radius, 2.5 mile radius, 3.0 mile radius, 3.5 mile radius, 4.0 mile radius, 4.5 mile radius, 5.0 mile radius, etc.). In at least some configurations, another assessment can be made (either concurrently or sequentially to the geographic assessment) to determine whether a pattern exists. Once a geographic link is established between the first and one or more of a second or subsequent user, or users, a comparison of user profile data and/or trending data is performed to identify other users having similar profiles and/or trending data positioned in the same geographic region(s). Additionally, an assessment can be made of a historical response and/or historical trends by users to similar conditions and/or similar geographies. An alert is then generated advising those users, as well as any other users on the network, to be aware that conditions exist that may cause them to have an episode. The alert can also be configured to provide specific suggestions for action by the users. The alert can be sent to the users via email, text message, pop-up, or any other mechanism selected by the user.

### Example 2

A first user reports to the handheld device (e.g., by entering text, responding to periodic inquiries, or interfacing with a device that takes a biological measurement), that there has been a condition change, e.g., a condition change to high risk. The report includes geographic positioning data, such as a GPS tag. The status of the first user is uploaded to a server via a network. A second user reports that there has been a condition change and the second user report is uploaded to a server via a network. An assessment is then made that both the first and second users are located within a geographic area set by the system for that area (e.g., 0.5 mile radius, 1 mile radius, 1.5 mile radius, 2 mile radius, 2.5 mile radius, 3.0 mile radius, 3.5 mile radius, 4.0 mile radius, 4.5 mile radius, 5.0 mile radius, etc.). Once a geographic link is established between the first and second user a comparison of user profile data is performed to identify other users having similar profiles positioned in the same geographic region. Additionally, an assessment can be made of a historical response by users to similar conditions. An alert is then generated advising the users to be aware that conditions exist that may cause them to have an episode. The alert can also be configured to provide specific suggestions for action by the users. The alert can be sent to the users via email, text message, pop-up, or any other mechanism selected by the user.

### Example 3

A first user reports to the handheld device (e.g., by entering text and/or interfacing with a device that takes a biological measurement and/or responding to periodic queries), that there has been a condition change, e.g., a condition change to high risk. The report includes geographic positioning data, such as a GPS tag. The status of the first user is uploaded to a server via a network. A second user reports that there has been a condition change and the second user report is uploaded to a server via a network. An assessment is made that both users are located within a geographic area set by the system (e.g., 0.5 mile radius, 1 mile radius, 1.5 mile radius, 2 mile radius, 2.5 mile radius, etc.). The system determines that both users are located within a geographic area that is a forest. An assessment is made of current environmental triggers in the geographic area. An assessment is made of users in the network who are, based on GPS positioning, approaching the area. An alert is then generated advising the users to be aware that conditions exist that may cause them to have an episode in the area they are approaching. The alert can also be configured to provide specific suggestions for action by the users or can be in the form of a personalized forecast for the user. The alert can be sent to the users via email, text message, pop-up, or any other mechanism selected by the user.

In some configurations, the alert can compare conditions to a prior incident experienced by the user to give additional context to the user.

### Example 4

A first user's handheld electronic device sends GPS location coordinates to the network via a communication network. Based on the location, data is retrieved about environmental conditions including, but not limited to, local weather, air quality, and pollen count. A report of predicted probability of experiencing a respiratory episode is developed and provided via the communication network to the user. The report can be based on the user's history, the user's profile, the probability of experiencing a problem based on other user's histories, or combinations thereof.

At the time of detecting a new location, the system can query the user to determine whether the change is temporary (e.g., a vacation) or permanent (e.g., a relocation). Additionally, for temporary changes, the system can query the length of time and provide information concerning environmental factors impacting respiratory function based on known or historical data.

### Example 5

A first user's handheld electronic device sends GPS location coordinates to the network via a communication network. The system keeps track of the user's history, including geographic location. When the user relocates, permanently or semi-permanently, to a new geographic area, the system sends a medication reminder to the handheld electronic device over the network. If a user relocates to a geographic location where a change in medication might be appropriate a notice can be delivered that identifies current medication and its optimal application, change in environmental factors, and a suggestion that the user visit his or her healthcare practitioner to ensure no change in medication or treatment protocol is appropriate due to the change in circumstances.

### Example 6

A first user provides information associated with the user of a rescue inhaler or other interventional procedures into a program accessible via an electronic device. The device associates a data and time stamp along with GPS data and environmental information from the device or from third party sources. The information is analyzed to identify potential triggers for the patient. When the system detects that the user is in conditions approaching those associated with an earlier incident, an alert is generated to facilitate the patient's ability to take evasive behavioral steps to avoid or minimize the likelihood of a respiratory episode.

### Example 7

GPS data for a user indicates that the user is traveling at a rate of 60 MPH and is approaching an area with a high pollen count. The system is adaptable to consider data on the rate of change of location with a projected destination (or a destination provided by a user input) and provides a dynamic projection to the user of the likelihood of experiencing a respiratory episode. Information can be analyzed and refreshed at a rate or frequency determined from the rate of change of location, elevation, or mere passage of time.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understothat various alternatives to the embodiments of the invention described herein may be employed in practicing the invention. It is intended that the following claims define the scope of the invention and that methods and structures within the scope of these claims and their equivalents be covered thereby.

1. A respiratory device comprising:
a. a housing adaptable and configurable to communicate with an electronic device;
b. a mouth piece having a proximal end and a distal end configurable to engage a mouth of a patient and transmit an air flow;
c. one or more diaphragm sensors configured to detect a breath vibration from the air flow in the mouth piece; and
d. a processor adaptable and configurable to analyze the breath vibration detected by the one or more diaphragm sensors.

2. The respiratory device of claim 1 wherein the respiratory device is adapted and configured to measure one or more of peak expiratory flow-rate, peak inspiratory flow-rate, mean flow-rates, volumes, flow over time, Forced Vital Capacity, percentage of flow at certain time intervals, and slow and forced volumes at certain time intervals, the device.

3. The respiratory device of claim 1 wherein the respiratory device is in communication with a display adapted and configured to display a result of an analysis of the breath vibration.

4. The respiratory device of claim 1 further comprising: a windscreen positionable relative to the mouth piece to reduce a secondary air flow from entering the mouth piece.

5. The respiratory device of claim 1 in communication with an environmental data source.

6. The respiratory device of claim 5 wherein the environmental data source is an environmental sensor associated with one or more of the respiratory device and the electronic device.

7. The respiratory device of claim 1 wherein the processor is further adaptable and configurable to correlate breath vibration data and environmental data.

8. The respiratory device of claim 1 wherein the processor generates an asthma output.

9. The respiratory device of claim 1 wherein the respiratory device is in communication with an audible indicator.

10. A system comprising:
a. a measurement probe, the probe comprising;
i. one or more diaphragm sensors adaptable and configurable to detect a breath vibration;
ii. a microphone;
iii. a mouth piece positioned proximally to the microphone; and
iv. a port,
b. a computing system adaptable to engage the port and having a computer executable instruction that, when executed by a processor, performs a vibration analysis of the diaphragm vibration and display the result.

11. The system of claim 10 wherein the system is adapted and configured to measure one or more of peak expiratory flow-rate, peak inspiratory flow-rate, mean flow-rates, volumes, flow over time, Forced Vital Capacity, percentage of flow at certain time intervals, and slow and forced volumes at certain time intervals, the device.

12. The system of claim 10 wherein the respiratory device is in communication with a display adapted and configured to display a result of an analysis of the breath vibration.

13. The system of claim 10 further comprising: a windscreen positionable relative to the mouth piece to reduce a secondary air flow from entering the mouth piece.

14. The system of claim 10 in communication with an environmental data source.

15. The system of claim 14 wherein the environmental data source is an environmental sensor associated with one or more of the respiratory device and the electronic device.

16. The system of claim 10 wherein the processor is further adaptable and configurable to correlate breath vibration data and environmental data.

17. The system of claim 10 wherein the processor generates an asthma output.

18. The respiratory device of claim 10 wherein the respiratory device is in communication with an audible indicator.

19. A system comprising:
a. an electronic device having a microphone adaptable and configurable to be in communication with a communication network;
b. a housing attachable to the electronic device, the housing further comprising:
i. a mouth piece aligned with a microphone of the electronic device; and
ii. an optional windscreen resided in a mouth piece tube;
c. a computer executable instruction that, when executed by a processor performs operations including vibration analysis of the microphone diaphragm of the probe and further adaptable to instruct that a result of the vibration analysis be displayed on a display.

20-28. (canceled)

29. A non-transitory computer readable medium storing instructions that, when executed by a computing device, causes the computing device to perform a method, the method comprising:
receiving one or more of each of a peak expiratory flow-rate, a peak inspiratory flow-rate, and a spirometry measurement from a respiratory device having a housing adaptable and configurable to communicate with an electronic device to receive a user respiratory input;
at least one or more of analyzing, monitoring, evaluating, and responding to the received one or more of the peak expiratory flow-rate, the peak inspiratory flow-rate, and the spirometry measurement.

30-38. (canceled)

39. A computing device comprising:
a processor configured to:
receive one or more of each of a peak expiratory flow-rate, a peak inspiratory flow-rate, and a spirometry measurement from respiratory device having a housing adaptable and configurable to communicate with an electronic device to receive a user respiratory input;
at least one or more of analyze, monitor, evaluate, and respond to the received one or more of the peak expiratory flow-rate, the peak inspiratory flow-rate, and the spirometry measurement.

40-49. (canceled)

49. A method comprising:
receiving one or more of each of a peak expiratory flow-rate, a peak inspiratory flow-rate, and a spirometry measurement from a respiratory device having a housing adaptable and configurable to communicate with an electronic device to receive a user respiratory input;
receiving an environmental data input;
at least one or more of analyzing, monitoring, evaluating, and responding to the received one or more of the peak expiratory flow-rate, the peak inspiratory flow-rate, and the spirometry measurement.

50-58. (canceled)

59. A method comprising:
transmitting, via a user computing device one or more of each of a peak expiratory flow-rate, a peak inspiratory flow-rate, and a spirometry measurement from a respiratory device having a housing adaptable and configurable to communicate with an electronic device to receive a user respiratory input to a web-server over a network;
obtaining an environmental data input;
at least one or more of analyzing, monitoring, evaluating, and responding to the received one or more of the peak expiratory flow-rate, the peak inspiratory flow-rate, and the spirometry measurement.

60-68. (canceled)

69. A system comprising:
a. an electronic device configurable to be in communication with a communication network;
b. a computer executable instruction that, when executed by a processor determines a likelihood of a respiratory event based on one or more of each of historical patient data, patient data input, current environmental data, current data for other patients in a similar geographic location and historical data for other patients in a similar geographic location.

70-78. (canceled)

79. A non-transitory computer readable medium storing instructions that, when executed by a computing device, causes the computing device to perform a method, the method comprising:
receiving one or more of a GPS location and a condition indication for each of one or more patients comprising a patient group;
at least one or more of analyzing, monitoring, evaluating, and providing a prediction for a second patient based on the GPS location of the second patient and at least one or more of the GPS location and condition indication for the one or more patients comprising the patient group.

80-88. (canceled)

89. A computing device comprising:
a processor configured to:
receive one or more of GPS location and condition indication for each of one or more patients comprising a patient group;
at least one or more of analyze, monitor, evaluate, and provide a prediction for a second patient based on the GPS location and at least one or more of the GPS location and condition indication for the one or more patients comprising the patient group.

90-98. (canceled)

99. A method comprising:
receiving one or more of a GPS location and a condition indication for each of one or more patients comprising a patient group;
at least one or more of analyzing, monitoring, evaluating, and providing a prediction for a second patient based on the GPS location of the second patient and at least one or more of the GPS location and the condition indication for the one or more patients comprising the patient group.

100-108. (canceled)
